(19) United States
(12) Patent Application Publication
(10) Pub. No.: US 2003/0007939 A1
(43) Pub. Date: Jan. 9, 2003

(54) Pharmaceutical compositions and methods for managing dermatological conditions

(76) Inventor: Howard Murad, Marina del Rey, CA (US)

Correspondence Address: PENNIE & EDMONDS LLP, 1667 K STREET NW, SUITE 1000, WASHINGTON, DC, 20006

(21) Appl. No.: 10/077,928

(22) Filed: Feb. 20, 2002

Related U.S. Application Data
(63) Continuation-in-part of application No. 09/953,431, filed on Sep. 17, 2001.
Application No. 09/953,431 is a continuation-in-part of application No. 09/878,231, filed on Jun. 12, 2001, now Pat. No. 6,383,523.
Application No. 09/878,231 is a continuation of application No. 09/549,202, filed on Apr. 13, 2000, now Pat. No. 6,296,880.
Application No. 09/549,202 is a continuation-in-part of application No. 09/330,127, filed on Jun. 11, 1999, now Pat. No. 6,071,541.
(60) Provisional application No. 60/094,775, filed on Jul. 31, 1998.

(51) Int. Cl. A61K 33/40; A61K 7/04; A61K 7/06

(52) U.S. Cl. 424/61; 424/70.1; 424/616

(57) ABSTRACT

Pharmaceutical composition including hydrogen peroxide and at least one other dermatological agent. The pharmaceutical compositions cleanse the dermatological surfaces and facilitate penetration of the at least one other dermatological for the treatment of a dermatological condition. The pharmaceutical compositions are useful in methods of treating, preventing, and managing skin conditions, scalp conditions, hair conditions, and nail conditions.

### CROSS-REFERENCE TO RELATED APPLICATIONS

This application is a continuation-in-part of application Ser. No. 09/953,431, filed Sep. 17, 2001, currently pending, which is a continuation-in-part of application Ser. No. 09/878,231, filed Jun. 12, 2001, now allowed, which is a continuation of application Ser. No. 09/549,202, filed Apr. 13, 2000, currently U.S. Pat. No. 6,296,880, which is a continuation-in-part of application Ser. No. 09/330,127, filed Jun. 11, 1999, currently U.S. Pat. No. 6,071,541, which is a continuation-in-part of provisional Application No. 60/094,775, filed Jul. 31, 1998.

### TECHNICAL FIELD

This application relates to pharmaceutical compositions and methods for preventing, treating, and managing dermatological conditions.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by, for example, dryness, yeast, and structural changes in the skin, such as due to aging and excessive sun exposure.

The scalp is the skin and subcutaneous tissue covering the neurocranium. The scalp encompasses a collection of hair follicles capable of hair production. These hairs are filamentous growths composed primarily of keratin and other proteins which extend from the dermis through the epidermis and out of the scalp.

Nails are the horny cutaneous plates on the dorsal surfaces of the distal ends of fingers or toes. The nail consists of the corpus or body (the visible part) and the radix or root at the proximal end concealed under a fold of the skin. The underpart of the nail is formed from the stratum germanativum of the epidermis, the free surface from the stratum lucidum, the thin cuticular fold overlapping the lunula representing the stratum corneum. Although nail material is similar to the stratum corneum, being derived from epidermis, it is composed primarily of hard keratin, which is highly disulfide-linked, and is approximately 100-fold thicker than stratum corneum.

Various pharmaceuticals have been used for the treatment or prevention of conditions of the skin, scalp, hair, and nails. Some of these compositions are discussed below.

Canadian Patent No. 1,174,976 discloses a germ-killing skin medication including two gels to be applied and mixed in situ, the first gel having sodium chlorite in an aqueous form and the second gel having lactic acid in an aqueous gel.

Great Britain Application No. 2,076,286 A discloses a dermatological composition of an oil medium dispersed in an aqueous medium that contains hydrogen peroxide, a buffer to maintain the composition below a pH of 7, and a starch gelled in situ. The buffer may include lactic, citric, tartaric, maleic, or hydroxysuccinic acids with an acid salt.

Great Britain Application No. 2,189,394 A discloses a concentrate that can be mixed with hydrogen peroxide to become an effective disinfectant for water, foodstuff, animal feeds, equipment, packages, and the like. The concentrate includes an inorganic acid with a pH less than 1.6, a silver compound or colloidal silver, an organic acid stabilizer such as tartaric, lactic, salicylic, or citric acid, and optionally gelatin.

European Patent Application No. 0,191,214 A2 discloses a cosmetic liquid cleanser for treating blemished, scarred, or inflamed skin having boric acid or borax, ammonium hydroxide, a peroxide, and optionally salicylic acid.

European Patent No. 0,250,539 B1 discloses a stabilized aqueous hydrogen peroxide composition having 0.1 to 4 weight percent hydrogen peroxide and 0.5 to 5 weight percent β-crystals of one or more lipids selected from monoglycerides of fatty acids, ascorbic acid, phosphate or lactic acid esters of fatty acids and monoglycerol ethers, said fatty acids and ether chains being saturated and having 12 to 18 carbons.

European Patent No. 0,425,507 B1 discloses compositions for treating abnormal or damaged conditions of the epithelium including skin, which include 0.01 to 12 weight percent of an activated protein containing at least 0.5 weight percent cysteine, 0.1 to weight percent of a reducing agent to reduce cystine to cysteine, and 81.0 to 99.889 weight percent water, acids, bases, buffering agents, emulsifying agents, thickeners, solvents, preservatives, coloring agents, and perfuming agents. The reducing agent may be a salt of a thioglycolic acid. In a preferred embodiment, the composition also includes an oxidizing agent, such as hydrogen peroxide.

U.S. Pat. No. 3,297,456 discloses cleaning and polishing compositions, particularly for floor waxing, having lactic acid, methanol, hydrogen peroxide, and aqua ammonia in a particular ratio.

U.S. Pat. Nos. 4,015,058 and 4,015,059 disclose stable peroxy-containing concentrates useful for the production of microbicidal agents consisting essentially of an aqueous mixture of 0.5 to 20 weight percent peracetic or perpropionic acid or their precursors, 25 to 40 weight percent hydrogen peroxide, and optionally up to 5 weight percent anionic surface-active compounds of the sulfonate and sulfate type. Also disclosed are compositions that further include 0.25 to 10 weight percent organic phosphonic acid capable of sequestering bivalent metal cations and their water-soluble acid salts.

U.S. Pat. No. 4,203,765 discloses an aqueous acidic etch-bleach solution of hydrogen peroxide, iron ions, and inorganic anions that form a silver salt, such that in the dissolved state the solution contains citric acid and a polymer of alkylene oxide units for stabilization of the hydrogen peroxide.

U.S. Pat. No. 4,438,102 discloses compositions containing gelatin, hydrogen peroxide, ammonium hydroxide, thioglycolic acid, and a lower alkanol to promote the growth of dermal and epidermal tissue.

U.S. Pat. No. 4,534,945 discloses an aqueous 25 to 35 weight percent solution of hydrogen peroxide stabilized against decomposition with up to 1.4 mg/L tin, which is maintained in solution by particular amounts of phosphate in the form of phosphonic acid and hydroxycarboxylic acid.

U.S. Pat. No. 4,557,935 discloses a germicidal composition of hydrophilic lipid crystals of 1-monolaurin, and preferably 1-monomyristin, and hydrogen peroxide, whereby the former stabilize the latter. Optionally, the compositions further contain salicylic acid.

U.S. Pat. No. 4,900,721 discloses liquid, aqueous disinfectants based on alcohol and hydrogen peroxide that contain one or more C₂₋₈ alcohols, hydrogen peroxide or a hydrogen peroxide forming compound, one or more carboxylic acids, one or more microbicidally active nitrogen-containing organic compounds, one or more microbicidally active phenolic compounds for disinfection of the skin and mucous membrane.

U.S. Pat. No. 5,139,788 discloses an antimicrobial surface sanitizing composition having a diluent and antimicrobial agent of an antimicrobially effective amount of alpha-hydroxyacid substituted mono- or di-carboxylic acid and an antimicrobially effective amount of hydrogen peroxide, such that the composition leaves a non-contaminating residue after contact with surfaces to be disinfected.

U.S. Pat. No. 5,693,318 discloses phosphate esters for the improvement of water solubility of salicylic acid and peroxide compounds in an aqueous cleanser.

U.S. Pat. No. 4,668,509 discloses polythioalkanecarboxylic anionic products and their preparation and use in cosmetic compositions and hair treatment compositions, such as shampoos.

U.S. Pat. No. 4,814,166 discloses polyanionic oligomer compounds suitable for use in keratin fiber treatment, such as hair, which may be administered in a shampoo. These compounds are alleged to hold hair with suppleness and without significant hardening of the hair.

U.S. Pat. No. 5,344,971 discloses mercapto acids, such as thiolactic acid (2-mercaptopropionic acid), that are used as reducing agents for the permanent reshaping of hair or for depilatory milks and creams.

An article entitled “Hydroxy Acids and Skin Aging” discloses the use of hydroxy and other acids as skin peels and emollients that can moisturize, stimulate, and exfoliate the skin. [Smith, W.,

Soap/Cosmetics/Chemical Specialties, pp. 54-58, 76, September, 1993.]. A study was conducted with hydroxy, keto, carboxylic, or dicarboxylic acids, including glycolic and salicylic acids, to determine long-term rejuvenating benefits. The study noted that higher pH formulations resulted in less stimulatory activity and lower irritation, and concluded that various non-hydroxy acids would be expected to deliver long-term rejuvenating benefits. Id. at 58.

One publication, WO 95/33438, discloses skin or hair care products having an agent acting cosmetically on the hair or skin, such as thioglycolic acid preferably with an ammonium salt. The agent is absorbed in a fibrous material containing amino groups, such as cellulose based-fiber containing polysilicic acid.

U.S. Pat. No. 5,422,370 discloses methods of using hydroxyacid or related compounds, such as alpha 2-hydroxypropanoic acid (lactic acid), for the treatment of wrinkles. These compounds are also disclosed to be effective for enhancing the topical effects of other cosmetic and pharmaceutical agents for treatment of conditions such as dry skin, ichthyosis, eczema, palmar and plantar hyperkeratoses, dandruff, acne, pruritis, psoriasis, Darier's disease, lichen simplex chronicus, and warts. Similarly, U.S. Pat. No. 5,547,988 discloses methods for reducing the appearance of skin changes associated with aging, such as wrinkles, by topically applying a compound of glycolic acid, lactic acid, citric acid, or a salt thereof.

U.S. Pat. No. 5,587,149 discloses improved stable emulsions of polyethylene glycol-in-oil for topical application to the skin that contain one or more water soluble active ingredients, such as Vitamin C, glycolic acid, and the like.

Despite these references, there is still a need for pharmaceutical compositions and methods for the prevention, treatment, and management of skin, scalp, hair, and nail conditions. The present invention advantageously provides pharmaceutical compositions, as well as methods, for the prevention, treatment, and management of these conditions.

### SUMMARY OF THE INVENTION

The present invention relates to a pharmaceutical composition for treating, preventing, or managing a dermatological condition selected from the group consisting of a scalp condition, a hair condition, and a nail condition comprising: hydrogen peroxide in an amount sufficient to cleanse a dermatological surface without substantial irritation thereof; a moisturizing agent in an amount sufficient to facilitate hydration of the dermatological surface or prevent moisture loss from the dermatological surface; and one or more dermatological agents selected from an antimicrobial agent in an amount sufficient to inhibit microorganisms on the scalp, hair, or nails; an anti-inflammatory agent in an amount sufficient to reduce inflamation of the scalp, hair, or nails; or a combination thereof.

In one preferred embodiment, the pharmaceutical composition is for treating scalp. In another preferred embodiment, the pharmaceutical composition is for treating hair. In yet another preferred embodiment, the composition is for treating nails.

In another preferred embodiment, the moisturizing agent is a hydrophobic agent, hydrophilic agent, or a combination thereof.

In one embodiment, the antimicrobial agent selected from an antibacterial, antifungal, antiviral, anthelmintic, or combinations thereof. In a preferred embodiment, the antimicrobial agent is single broad spectrum antimicrobial agent selected from echinacea, golden seal, benzalkonium chloride, benzethonium chloride, iodine, grape seed extract, pomegranate extract, green tea extract, polyphenols, and combinations thereof. In another preferred embodiment, the antibacterial is selected from triclosan, neomycin, polymyxin, bacitracin, clindamycin, benzoyl peroxide, a tetracycline, a sulfa drug, a penicillin, a quinolone, a cephalosporin, and combinations thereof. In another preferred embodiment, the antiviral is selected from acyclovir, tamivir, penciclovir, and combinations thereof. In another preferred embodiment, the antifungal is selected from farnesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and combinations thereof. In another preferred embodiment, the anthelmintic is metronidazole.

In another embodiment, the anti-inflammatory is a non-steroidal agent, a steroidal agent, or a combination thereof. In a preferred embodiment, the steroidal agent is selected from hydrocortisone, fluocinolone acetonide, halcinonide, halobetasol propionate, clobetasol propionate, betamethasone dipropionate, betamethasone valerate, triamcinolone acetonide, and combinations thereof. In another preferred embodiment, the non-steroidal agent is selected from aspirin, ibuprofen, ketoprofen, naproxen, aloe vera gel, aloe vera, licorice extract, pilewort, Canadian willow root, zinc, allantoin, and combinations thereof.

Advantageously, the pharmaceutical composition further comprises an amount of amphoteric surfactant and an amount of citric acid sufficient to inhibit decomposition of hydrogen peroxide for at least three months. In a preferred embodiment, the amount of amphoteric surfactant and citric acid is sufficient to inhibit hydrogen peroxide decomposition at 40° C. for at least three months. In a more preferred embodiment, the hydrogen peroxide is present in an amount from about 0.01 to 6 weight percent of the composition and the moisturizing agent is present in an amount from about 0.01 to 20 weight percent of the composition.

In one embodiment, the pharmaceutical composition further comprises a pharmaceutically acceptable carrier or excipient. In another embodiment, the pharmaceutical composition further comprises an amount of Retinol, Vitamin K, Arnica Montana, an immuno-enhancer, anti-oxidant, an extract of pomegranate, an extract of Morinda citrifolia, and combinations thereof.

In another embodiment, the pharmaceutical composition further comprises an exfoliant. In a preferred embodiment, the exfoliant is an enzymatic exfoliant. In a more preferred embodiment, the exfoliant is an acidic exfoliant.

The invention further relates to a method of treating, preventing, or managing a dermatological condition selected from the group consisting of a scalp condition, a hair condition, and a nail condition in a patient which comprises administering to the patient a therapeutically effective amount of a composition comprising: hydrogen peroxide in an amount sufficient to cleanse the surface of the scalp, hair, or nails without substantial irritation thereof; a moisturizing agent in an amount sufficient to facilitate hydration of the dermatological surface or prevent moisture loss from the dermatological surface; and one or more dermatological agents selected from an antimicrobial agent in an amount sufficient to at least inhibit microorganisms on the scalp, hair, or nails; an anti-inflammatory agent in an amount sufficient to reduce inflamation of the scalp, hair, or nails; or a combination thereof for treating, preventing, or managing the dermatological condition.

The invention also relates to a method of treatment wherein the antimicrobial agent is selected from an antibacterial, antifungal, antiviral, an anthelmintic, and combinations thereof. The types of dermatological conditions that can be treated include psoriasis, folliculitis, rosacea, nail fungus, perioral dermatitis, seborrheic dermatitis, dandruff, or impetigo. The administration of the components may be topical, such as by a gel, paste, cream, lotion, emulsion, shampoo, or ointment. About 1 mg to 20,000 mg of the composition can be administered to achieve satisfactory results in most cases. In a preferred embodiment, the hydrogen peroxide, the moisturizing agent, and the one or more dermatological agents are administered concurrently. In another preferred embodiment, the hydrogen peroxide, the moisturizing agent, and the one or more dermatological agents are administered concurrently with at least one additional pharmaceutical composition for the prevention, treatment, or management of a dermatological condition. In a more preferred embodiment, the at least one additional pharmaceutical composition is selected from yellow dock, bupleurum, poria cocos, gentian root, myrr gum, hawthorn berry extract, rosemary extract, wild yam root, wild yam extract, marshmallow root, black cohosh, soy, ginger, an anti-oxidant, an immuno-enhancer, and combinations thereof.

### DETAILED DESCRIPTION OF THE PREFERRED EMBODIMENTS

A pharmaceutical composition for the prevention, treatment, and management of dermatological conditions has now been discovered. Moreover, the management of these dermatological conditions may advantageously be accomplished by the administration of the pharmaceutical composition of the present invention.

The term “dermatological conditions,” as used herein, means any combination of skin conditions, scalp conditions, hair conditions, or nail conditions, present any where on the skin, scalp, hair, or nails, caused by aging or extrinsic factors such as, but not limited to, sunlight, radiation, air pollution, wind, cold, dampness, heat, chemicals, smoke, and smoking. In one embodiment the dermatological conditions is a skin condition. In another embodiment the dermatological condition is a scalp condition. In another embodiment the dermatological condition is a hair condition. In yet another embodiment the dermatological condition is a nail condition.

The term “skin conditions,” as used herein, means a condition present any where on the skin including, but not limited to, pruritus; spider veins; lentigines; age spots; senile purpura; warts; keratosis; melasmas; blotches; wrinkles; nodules; atrophy; precancerous lesions; sun damaged skin; dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions; and the like.

The term “scalp conditions,” as used herein, means a condition present any where on the scalp including, but not limited to, dandruff, seborrhea, dry scalp, psoriasis and the fungus Pityrosporum ovale which tends to be present in patients with dandruff or seborrhea.

The term “hair conditions,” as used herein, means conditions present any where on the hair including, but not limited to, hair damage such as hair breakage and weathering damage; thinning of hair; and chemically processed hair, such as colored hair or straightened hair.

The term “nail conditions,” as used herein, means conditions present any where on the nails including, but not limited to, nail breakage, weathering damage, brittle nails, yellowing nails, paronychia, nail fungus or onychomycosis, and cracked nails.

The term “dermatological surface,” as used herein, means the surface of the skin, scalp, hair, or nails.

The terms “managing” or “management,” as used herein, includes one or more of the prevention, treatment, or modification of a skin condition.

The pharmaceutical composition of the invention includes hydrogen peroxide in an amount sufficient to cleanse the skin, scalp, hair, or nails and at least one other dermatological agent.

The hydrogen peroxide is present in an amount sufficient to cleanse at least a portion of the skin, scalp, hair, or nails. “Cleanse” as used herein includes the removal of dirt, debris, air pollutants, desquamating cells, and cutaneous secretions of the dermatological surface. Preferably, the hydrogen peroxide is present in an amount to cleanse the skin, scalp, hair, or nails without substantial irritation. The hydrogen peroxide is typically present in an amount from about 0.01 to 6 weight percent, preferably 0.05 to 4 weight percent, and more preferably 0.1 to 1 weight percent of the composition. Without wishing to be bound by theory it is believed that cleansing the skin, scalp, hair, or nails with hydrogen peroxide improves penetration of the one or more dermatological agents into the skin, scalp, hair, or nails and, thus, improves the efficacy of the one or more other dermatological agents.

In a preferred embodiment, the pharmaceutical compositions of the invention further includes one or more moisturizing agents. “Moisturizing agent,” as used herein, is used to include any agent that facilitates hydration of the skin, scalp, hair, or nails by inhibiting or preventing loss of water from the skin, scalp, hair, or nails; absorbs water from the atmosphere and hydrates the skin, scalp, hair, or nails; or enhances the ability of the skin, scalp, hair, or nails to absorb water directly from the atmosphere; or a combination thereof. Without wishing to be bound by theory it is believed that the moisturizing agent further improves the skin, scalp, hair, or nails ability to absorb the at least one or more dermatological agents. Furthermore, moisturizing agents also minimize or prevent the skin, scalp, hair, or nails from drying and cracking; cracked skin, scalp, hair, and nails is more susceptible to environmental factors that generate free radicals, which are believed to cause further damage to the skin, scalp, hair, or nails. Suitable moisturizing agents include, but are not limited to, hydrophobic agents, and hydrophilic agents, or combinations thereof. Moisturizers, when used, are typically present in an amount from about 0.01 to 20 weight percent, preferably about 0.05 to 10 weight percent, more preferably from about 0.1 to 5 weight percent of the composition.

Moisturizing agents that are hydrophobic agents include, but are not limited to, ceramide, borage oil (linoleic acid), tocopherol (Vitamin E), tocopherol linoleate, dimethicone, glycerine, and mixtures thereof. Hydrophobic agents, when present, are believed to moisturize the skin, scalp, hair, or nails by inhibiting or preventing the loss of water from the skin, scalp, hair, or nails. The hydrophobic agent, when present, is typically present in an amount from about 0.01 to 20 weight percent, preferably from about 0.05 to 15 weight percent, and more preferably from about 0.1 to 5 weight percent of the composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin, scalp, hair, or nails by absorbing moisture from the atmosphere to hydrate or facilitate hydration of the skin. The hydrophilic agent, when present, is typically present in an amount from about 0.01 to 20 weight percent, preferably from about 0.05 to 15 weight percent, and more preferably from about 0.1 to 5 weight percent of the composition.

Other moisturizing agents that hydrate the skin, scalp, hair, or nails, and are useful in the compositions and methods of the present invention include, but are not limited to, panthenol; primrose oil; GLA 3 and other fish oils that may include, for example, the omega-3 and omega-6 oils and/or linoleic acid; and flax seed oil. Preferably, these moisturizing agents are administered orally.

Without wishing to be bound by theory it is believed that the hydrogen peroxide and the moisturizing agent of the invention interact in a synergistic manner to provide the desired management of the skin, scalp, hair, or nails. The hydrogen peroxide cleanses the skin, scalp, hair, or nails and removes substances foreign to the skin, scalp, hair, or nails and improves penetration of the one or more other dermatological agents into the skin, scalp, hair, or nails. The moisturizing agent moisturizes the skin, scalp, hair, or nails and further improves penetration of the one or more other dermatological agents into the skin, scalp, hair, or nails. The synergistic effect provides enhanced efficacy in treating dermatological conditions.

The at least one other dermatological agent may be any other dermatological agent known to those of ordinary skill in the art for treating a dermatological condition. For example, the at least one other dermatological agent may be one or more of Arnica Montana (a healing herb); any vitamin A source including retinyl palmitate or other retinyl esters, retinoic acid, or Retinol; and Vitamin K. The Arnica Montana facilitates skin healing and acts as an antiseptic and local anti-inflammatory, and, when used, is typically present in an amount from about 0.1 to 2 weight percent, preferably about 0.2 to 1 weight percent. The Retinol facilitates normal skin, hair, and nail production, particularly epidermal normalization, and, when used, is typically present in an amount from about 0.01 to 6 weight percent, preferably about 0.1 to 5 weight percent. The Vitamin K inhibits or suppresses inflammation and bruising (i.e., acts as an anti-inflammatory and anti-bruising agent) and, when used, is typically present in an amount from about 0.01 to 1 weight percent, preferably from about 0.1 to 0.5 weight percent.

In a more preferred embodiment, the dermatological agent further includes an exfoliant to help remove dead or dying cells from the skin, scalp, hair, or nails and further improve the skin, scalp, hair, or nail's own ability to absorb moisture directly from the atmosphere. Preferably, the exfoliant is used in combination with one or more hydrophilic agents to help absorb moisture from the atmosphere and hydrate the skin or in combination with one or more hydrophobic agents to inhibit or prevent moisture loss by the skin. More preferably, thepharmaceutical composition includes one or more of ahydrophilic agent and one or more of a hydrophobic agent in combination with an exfoliant. It is believed that the exfoliant also helps the one or more other dermatological agents penetrate the skin, scalp, hair, or nails.

The exfoliant may be an enzymatic exfoliant, or an acidic exfoliant. Any enzymatic exfoliant known to those skilled in the art may be used in the compositions and methods of the invention. Examples of enzymatic exfoliants useful in the compositions and methods of the invention include, but are not limited to, papain, from papaya, and bromalein, from pineapple.

Examples of acidic exfoliants include, but are not limited to a mono- or poly-hydroxy acid, tannic acid, or a mixture thereof, or a pharmaceutically acceptable salt or ester thereof. One of ordinary skill in the art will be readily able to select and prepare suitable mono- or poly-hydroxy acids for use in the composition of the invention, for example, alkyl hydroxycarboxylic acids, aralkyl and aryl hydroxycarboxylic acids, polyhydroxy-carboxylic acids, and hydroxy-polycarboxylic acids. One of ordinary skill in the art would typically select one or more of the following mono- or poly-hydroxy acids: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof, and 2-hydroxyphenyl pyruvic acid and esters thereof.

In one embodiment the hydroxy acidic component is an alpha-hydroxy acid. Preferred alpha-hydroxy acids include citric acid, glycolic acid, lactic acid. In another embodiment the hydroxy acidic exfoliant is a beta-hydroxy acid. A preferred beta-hydroxy acid is salicylic acid.

The term “pharmaceutically acceptable salt” refers to a salt prepared from pharmaceutically acceptable non-toxic acid. Examples of suitable inorganic metallic bases for salts formation with the acid compounds of the invention include, but are not limited to, aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

It should be understood that one or more derivatives of the above acidic component, such as esters or lactones thereof, are also suitably used. One of ordinary skill in the art will also understand that various hydroxy acids described in U.S. Pat. Nos. 5,547,988 and 5,422,370 are also suitable for use in the compositions and methods of the invention. The acidic component is present in the composition and methods in an amount sufficient to exfoliate, i.e., remove dead or dying cells, from at least a portion of the skin, scalp, hair, or nails. The acidic component is typically present in an amount from about 0.1 to 12 weight percent, preferably about 1 to 11 weight percent, more preferably from about 4 to 10 weight percent of the composition. For example, the acidic component may be from about 0.1 to 3 weight percent citric acid in combination with up to about 2 weight percent salicylic acid.

Application of chemicals to the hair, such as for coloring or other hair treatment, requires the cuticles to be opened, which may be accomplished by using any of a variety of alkaline components suitable for application to the hair. It should be understood that the application of the acidic component, however, will close or essentially close the cuticle incidental to the methods of managing scalp conditions, as discussed herein. Thus, chemical processing of the hair is rendered more difficult after application of the compositions of the present invention. However, when it is desired to specifically protect chemically processed hair, the compositions herein may be administered subsequent to, preferably immediately subsequent to, chemical processing of the hair. By “immediately” it is meant that the compositions herein are administered within 4 hours, preferably within 1 hour, and more preferably within 30 minutes of the chemical processing. Thus, in a preferred embodiment for protecting chemically processed hair, a patient may optionally have their hair conventionally shampooed and rinsed, chemically processed, and then have the compositions of the invention administered to protect the processing by closing or essentially closing the cuticle.

The at least one other dermatological may also preferably include one or more anti-inflammatory components in an amount sufficient to reduce redness and swelling of the skin, an immuno-enhancer component in an amount sufficient to boost the immune system to facilitate repair of damaged skin, scalp, hair, or nails, and one or more additional antioxidants in an amount sufficient to neutralize free radicals, or a combination thereof.

The one or more anti-inflammatory agents is present in an amount sufficient to reduce inflammation of the skin. In one embodiment the anti-inflammatory agent is a steroidal anti-inflammatory. Suitable steroidal anti-inflammatory agents for use in the compositions and methods of the invention include the corticosteroids such as, but not limited to, hydrocortisone, fluocinolone acetonide, halcinonide, halobetasol propionate, clobetasol propionate, betamethasone dipropionate, betamethasone valerate, and triamcinolone acetonide.

In another embodiment the anti-inflammatory agent is a non-steroidal anti-inflammatory agent. Examples of suitable non-steroidal anti-inflammatory agents for use in the compositions and methods of the invention include, but are not limited to, aspirin, ibuprofen, ketoprofen, and naproxen. These anti-inflammatory agents are preferably administered orally. Other non-steroidal anti-inflammatory agents useful in the compositions of the invention include, but are not limited to, aloe vera gel, aloe vera, licorice extract, pilewort, Canadian willow root, zinc, and allantoin. Allantoin is a preferred non-steroidal anti-inflammatory agent. The anti-inflammatory agents are used in an amount sufficient to inhibit or reduce inflammation, preferably in an amount from about 0.02 to 2 weight percent, preferably from about 0.1 to 1.5 weight percent, and more preferably from about 0.2 to 1 weight percent of the composition. It should be understood, with reference to managing dermatological conditions, that the anti-inflammatory agents facilitate inhibition or suppression of inflammation any where on the skin or scalp. Arnica Montana (a healing herb) and vitamin K can also be used as the anti-inflammatory. Arnica Montana facilitates skin healing and acts as an antiseptic and local anti-inflammatory, and, when used, is typically present in an amount from about 0.1 to 2 weight percent, preferably about 0.2 to 1 weight percent. Vitamin K inhibits or suppresses inflammation and bruising (i.e., acts as an anti-inflammatory and anti-bruising agent) and, when used, is typically present in an amount from about 0.01 to 1 weight percent, preferably from about 0.1 to 0.5 weight percent.

In another preferred embodiment, the at least one other dermatological agent further comprises a pharmaceutically acceptable antimicrobial agent. Any pharmaceutically acceptable antimicrobial agent available to those of ordinary skill in the art may be used, but preferably at least one of an antibacterial agent, antifungal agent, antiviral agent, or anthelmintic will be used according to the invention. A single broad spectrum antimicrobial agent, i.e., one that is believed to have at least two of antibacterial, antifungal, and antiviral efficacy, including, but not limited to echinacea, golden seal, benzalkonium chloride, benzethonium chloride, iodine, grape seed extract, pomegranate extract, green tea extract or polyphenols, or combinations thereof, may be included. Another suitable antimicrobial agent includes the class of anthelmintics, such as metronidazole, to facilitate treatment of, e.g., tricomona infection. Preferred antiviral agents include, but are not limited to, acyclovir, tamivir, penciclovir, and the like, and mixtures thereof. Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof. Preferred antifungal agents include, but are not limited to, farnesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof. Exemplary tetracyclines include doxycycline and minocycline. An exemplary sulfa drug is sulfacetamide. An exemplary cephalosporin is cephalexin (commercially available as KEFLEX). Exemplary quinolones include the floxacins, such as loemfloxacin, ofloxacin, and triovafloxacin. It should be readily understood that any salts, isomers, pro-drugs, metabolites, or other derivatives of these antimicrobial agents may also be included as the antimicrobial agent in accordance with the invention. The antimicrobial agent is typically present in an amount from about 0.01 to 1.5 weight percent, preferably from about 0.1 to 1.2 weight percent, and more preferably from about 0.3 to 1 weight percent of the composition. The antimicrobial agent inhibits the formation of, and may further reduce the presence of, microbes that cause redness, inflammation, and irritation of the skin, scalp, or nails.

The immuno-enhancer component is present in an amount sufficient to boost the immune system to facilitate repair of damaged skin, scalp, hair, or nails. Suitable immuno-enhancers useful in the compositions of the invention include, but are not limited to, interferon, Aldara (Immiquimod), resiquimod, and tacrolimus (Prograf). The immuno-enhancer may be present in an amount from about 0.1 to 10 weight percent, preferably from about 0.5 to 5 weight percent of the composition.

Anti-oxidants of both the enzymatic and non-enzymatic type may be included in the compositions and methods of the invention. For example, superoxide dismutase (SOD), catalase, and glutathione peroxidase are natural enzymatic anti-oxidants used by the body that may be supplemented with the compositions herein. Suitable non-enzymatic anti-oxidants include, but are not limited to, Vitamin E (e.g., tocopherol), Vitamin C (ascorbic acid), carotenoids, Echinacoside and caffeoyl derivatives, oligomeric proanthocyanidins or proanthanols (e.g., grape seed extract), silymarin (e.g., milk thistle extract, Silybum marianum), ginkgo biloba, green tea polyphenols, pomegranate extract, and mixtures thereof. Carotenoids are powerful anti-oxidants, and they include beta-carotene, canthaxanthin, zeaxanthin, lycopen, lutein, crocetin, capsanthin, and the like. Preferably, the anti-oxidant component includes Vitamin E, Vitamin C, or a carotenoid. When vitamin C component is used as an antioxidant it is preferably an ascorbic acid, or a pharmaceutically acceptable salt or ester thereof, and more preferably ascorbyl palmitate, dipalmitate L-ascorbate, sodium L-ascorbate-2-sulfate, or an ascorbic salt, such as sodium, potassium, and calcium, or mixtures thereof. When oral formulations of the pharmaceutical composition are used, it is preferred that a non-acidic form of vitamin C be used to reduce the stomach irritation that may occur when using an acidic form. The anti-oxidant component, when used, is present in an amount sufficient to inhibit or reduce the effects of free-radicals. The anti-oxidant component may be present in an amount from about 0.001 to 1 weight percent, preferably from about 0.01 to 0.5 weight percent of the composition.

The at least one other dermatological agent may be an extract of Morinda citrifolia or an extract of pomegranate. The extract of Morinda citrifolia or the extract of pomegranate may be obtained from any part of the plant including, for example, the fruit, the skin or rind of the fruit, the seeds, the bark, the leaves, the roots, the flowers, or the stem. The extract of Morinda citrifolia acts as an antioxidant. The extract of pomegranate acts to neutralize free radicals. The extract of Morinda citrifolia or the extract of pomegranate, when present, is present in an amount from about 0.01 to 80 weight percent, preferably from about 0.1 to 20 weight percent, and more preferably from about 0.5 to 10 weight percent.

The at least one other dermatological agent may be a niacin component and/or a 5-α reductase inhibitor. These components are useful in pharmaceutical compositions for treatment, prevention, or management of thinning hair. The niacin component (vitamin B₃) may be any pharmaceutically acceptable niacin component, preferably niacinamide or nicotinate, more preferably nicotinate. The niacin component should be present in an amount sufficient to facilitate improved blood circulation in the scalp, which may inhibit hair loss. The niacin component, when present, is present in an amount of from about 0.01 to 1 weight percent, preferably, from about 0.05 to 0.8 weight percent, and most preferably from about 0.1 to 0.5 weight percent.

The 5-α reductase inhibitor may be any pharmaceutically acceptable 5-α reductase inhibitor. For example, the 5-α reductase inhibitor may be one or more of finasteride or Saw Palmetto Extract. The 5-α reductase inhibitor should be present in an amount sufficient to inhibit conversion of testosterone in the scalp to dihydro-testosterone, the latter of which is believed to increase hair thinning. The 5-α reductase inhibitor, when present, is present in an amount of from about 0.01 to 1 weight percent, preferably, from about 0.05 to 0.5 weight percent, and most preferably from about 0.08 to 0.2 weight percent.

Optionally, the pharmaceutical compositions also include at least one herb from the group of yellow dock, bupleurum, poria cocos, gentian root, myrr gum, hawthorn berry extract, rosemary extract, wild yam root, wild yam extract, marshmallow root, black cohosh, soy, or ginger.

Yellow Dock, also known as Rumex crispus, is often used to treat skin disease, especially those involving some form of inflammation. The active constituents of yellow dock are believed to be rumicin and chrysarobin. Yellow Dock extract, when included, is typically present in the pharmaceutical compositions of the invention in an amount from about 1 to 30 weight percent, preferably from about 3 to 25 weight percent, and more preferably from about to 20 weight percent.

Bupleurum, also known as Bupleurum falactum, is known for its effect on the liver. The active constituents in bupleurum are believed to be furfurol, sterol, and bupleurumol. The bupleurum, when included, is typically present in the pharmaceutical compositions of the invention in an amount from about 1 to 20 weight percent weight, preferably about 2 to 15 weight percent, and more preferably from about 3 to 10 weight percent.

The active constituents in poria cocos, also known as Lycoperdon solidum, are tetracyclic titerpenic acids, polysaccharides, ergostol, choline, lipase, and protease. This herb is useful for reducing or eliminating excess fluids from the body. When included in the pharmaceutical compositions of the invention, it is typically present in an amount from about 1 to 20 weight percent, preferably from about 2 to 15 weight percent, and more preferably from about 3 to 10 weight percent.

The bitter glycosides in gentian root, also known as Gentian lutea, account for its use as a digestive bitter and liver disorder treatment. Gentian root, when included in the pharmaceutical compositions of the invention, is typically present in an amount from about 1 to 20 weight percent, preferably from about 2 to 15 weight percent, and more preferably from about 3 to 10 weight percent.

Myrrh, also known as Commiphora myrrha, has several oils, resins and gums that increase circulation and heart rate. Myrrh gum, when included in the pharmaceutical compositions of the invention, is typically present in an amount from about 1 to 20 weight percent, preferably from about 2 to 15 weight percent, and more preferably from about 3 to 10 weight percent.

Hawthorn berry extract, also known as Crataegus supplement, can also be added to the dermatological compositions. This herb is useful in the treatment of heart disease. Crategolic acid, citric acid, tartaric acid, glavone, glycosides, and vitamin C are the active constituents of hawthorne berries. The hawthorn berry extract, when included in the pharmaceutical compositions of the invention, is typically present in an amount from about 0.5 to 8 weight percent, preferably from about 0.6 to 6 weight percent, and more preferably from about 0.7 to 4 weight percent of the composition.

Rosemary contains aromatic oils that my assist with stomach disorders, and salicylic acid. When included in the pharmaceutical compositions of the invention, rosemary is typically present in an amount from about 0.5 to 8 weight percent, preferably from about 0.6 to 6 weight percent, and more preferably from about 0.7 to 4 weight percent of the composition. Wild yam possesses glycoside saponins and diosgenins, hormonal precursors to cortical steroids that may help to reduce pain. It is believed to assist with problems of the liver and gall bladder, as well. When included in the pharmaceutical compositions of the invention, wild yam is typically present in an amount from about 0.5 to 8 weight percent, preferably from about 0.6 to 6 weight percent, and more preferably from about 0.7 to 4 weight percent.

The marshmallow root, also known as Althea officinalis, acts as an anti-inflammatory. The mucilage in the herb soothes membranes, thereby reducing inflammation. When included in the pharmaceutical compositions of the invention, marshmallow root is typically present in an amount from about 0.5 to 8 weight percent, preferably from about 0.6 to 6 weight percent, and more preferably from about 0.7 to 4 weight percent of the composition.

Black cohosh acts as a natural estrogen supplement. Soy and ginger may act as an anti-oxidant and may act as a moisturizer to hydrate or facilitate hydration of the skin, hair, or nails. The amount of these herbs, when present in the pharmaceutical compositions of the invention, may be readily determined by one of ordinary skill in the art.

The invention also contemplates using stem-cell therapy to improve the skins ability to function. In one embodiment of the invention, stem-cells, preferably from the patients own body, are contacted with the patients skin, typically by injection. The stem cells then develop into new skin cells and, thus, improve the skins ability to function.

The pharmaceutical composition may further optionally include one or more of a cysteine component, magnesium component, manganese component, carotenoid component, selenium component, and copper component.

The optional cysteine component assists in thickening the dermis, supplementing of collagen and elastic tissue, and consequently, reduction of wrinkles and other skin conditions. The cysteine component, when used in the composition, is preferably N-acetyl cysteine, or a pharmaceutically acceptable salt thereof, and is then typically present in an amount from about 1 to 10 weight percent, preferably from about 2 to 8 weight percent, and more preferably from about 3 to 6 weight percent of the composition.

The optional manganese component is the co-factor used by the SOD found in mitochondria. The manganese component may be any manganese compound, or pharmaceutically acceptable salt thereof, but preferably is manganese ascorbate or a manganese ascorbic acid complex. The manganese, when present, is typically present in an amount from about 0.5 to 10 weight percent, preferably from about 1 to 8 weight percent and most preferably from about 5 to 7 weight percent, wherein the manganese is present in an amount from about 5 to 20 weight percent of a complex such as manganese ascorbate.

The copper component may also be included in the pharmaceutical composition, and may be any copper compound, or a pharmaceutically acceptable salt thereof. The copper component inhibits elastase and assists in treatment of elastic tissue defects. Preferably, the copper compound is copper sebacate. The copper, when included in the composition, is typically present in an amount from about 5 to 20 weight percent of the copper sebacate. The copper component is typically present in an amount from about 0.01 to 5 weight percent, preferably from about 0.02 to 3 weight percent, and more preferably from about 0.03 to 2 weight percent of the composition.

The magnesium component is also optional and may be any magnesium compound, or a pharmaceutically acceptable salt thereof, but preferably is magnesium ascorbate or magnesium ascorbic acid complex, wherein the magnesium is typically present in about 5 to 20 weight percent of the complex. The magnesium component, when included in the composition, is typically present in an amount from about 1 to 10 weight percent, preferably from about 3 to 8 weight percent, and more preferably from about 5 to 7 percent of the composition.

Additionally, a source of selenium may also be optionally added to the pharmaceutical composition. Any selenium compound, or a pharmaceutically acceptable salt thereof maybe used. When present, the selenium compound is preferably selenium complexed with an amino acid. More preferably, the selenium compound is L-selenomethionine, wherein the selenium is present in an amount from about 0.1 to 5 weight percent of the complex. The selenium, when included, is typically present in an amount from about 0.01 to 3 weight percent, preferably from about 0.05 to 2 weight percent, and more preferably from about 0.1 to 1 weight percent in the pharmaceutical composition.

The pharmaceutical compositions of the invention may also include one or more of a local analgesic or anesthetic, antiyeast agent, antiperspirant, antipsoriatic agent, antiaging agent, antiwrinkle agent, sun screen and/or sun blocking agent, skin lightening agent, depigmenting agent, vitamin, hormone, or retinoid.

The compositions of the invention may further include one or more surfactants, stabilizers, preservatives, coloring agents, water, buffering agents, emulsifying agents, thickeners, solvents, perfuming agents, and the like. Preferably, the water is deionized water. It should be understood that water includes the remainder of a given composition after other ingredients are determined. Although any pharmaceutically acceptable surfactant, stabilizer, preservative, coloring agent, buffering agents, emulsifying agents, thickeners, solvents, or perfuming agents may be used, certain compounds or mixtures are preferred as discussed below.

Preferred surfactants, including both the foaming and non-foaming type, including, but not limited to, sodium laureth sulfate, sodium laureth-13 carboxylate, disodium laureth sulfosuccinate, disodium cocoamphodiacetate, and the like, and mixtures thereof. More preferably, at least one amphoteric surfactant is included in the composition, such as disodium cocoamphodiacetate. The amphoteric surfactant, in combination with citric acid, inhibits hydrogen peroxide decomposition. The surfactant component may be present in an amount from about 10 to 90 weight percent, preferably about 20 to 80, and more preferably about 30 to 70 weight percent of the composition.

The term “inhibit hydrogen peroxide decomposition,” as used herein, means to at least stop the rate of decomposition from increasing, preferably to inhibit the decomposition entirely, and more preferably to substantially inhibit the decomposition altogether. “Substantially inhibit,” as used herein, means that less than about 10 weight percent, preferably less than about 3 weight percent, and more preferably less than about 1 weight percent, of the hydrogen peroxide decomposes over a three month period of time at 40° C.

A preferred stabilizer includes glycol stearate or PEG-150 distearate. The stabilizer, when used, is typically present in an amount from about 0.1 to 5 weight percent of the composition.

Preferred preservatives include tetrasodium ethylene-diamine tetraacetic acid (EDTA), methylparaben, benzophenone-4, methylchloroisothiazolinone, methylisothiazolinone, and the like, and mixtures thereof. Preservatives, when used, are typically present in an amount from about 0.01 to 6 weight percent, preferably about 0.05 to 4 weight percent, and more preferably from about 0.1 to 2 weight percent.

Preferred coloring agents include FD & C Green No. 3, FD & C Violet No. 2, FD & C Yellow No. 5, FD & C Red No. 40, and the like, and mixtures thereof The coloring agents, when used, are typically present in an amount from about 0.001 to 0.1 weight percent, and preferably from about 0.005 to 0.05 weight percent of the composition.

The pharmaceutical compositions of the invention may also include a pharmaceutically acceptable carrier. Any suitable pharmaceutically acceptable carrier readily apparent to those of ordinary skill in the art may be combined with the hydrogen peroxide and the at least one other dermatological agent, to provide the pharmaceutical compositions of the invention. Pharmaceutically acceptable carriers include, but are not limited to, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproply methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones. For example, preferred topical formulations of the pharmaceutical composition may include a silicon-containing carrier, but in amounts insufficient to cause substantial irritation. Suitable silicones include cyclomethicone or a mixture of cyclopentasiloxane and dimethicone/vinyldimethicone crosspolymer.

Those of ordinary skill in the art will also understand that topical effectiveness of pharmaceuticals requires percutaneous absorption and bioavailability to the target site. Thus, the compositions and methods of the invention require penetration through the stratum corneum into the epidermal layers of the skin or scalp or penetration into the surface of the hair or nails, as well as sufficient distribution to the sites targeted for pharmacologic action. To facilitate percutaneous absorption the pharmaceutical compositions may further include a “penetration enhancer.” The phrase “penetration enhancer,” as used herein, means any agent that enhances the penetration of the dermatological agent through the skin, scalp, hair, or nails.

Suitable penetration enhancers for use in the compositions and methods of the invention include, but are not limited, to urea, C₂-C₄ alcohols such as ethanol and isopropanol, polyethylene glycol monolaurate, polyethylene glycol-3-lauramide, dimethyl lauramide, sorbitan trioleate, fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-,di- and tri-glycerides of fatty acids. Suitable fatty acids include, but are not limited to lauric acid, myristic acid, stearic acid, oleic acid, linoleic acid and palmitic acid. Monoglyceride permeation enhancers include glycerol monooleate, glycerol monolaurate, and glycerol monolinoleate, for example. Examples of penetration enhancers useful in the methods of the invention include, but are not limited to those described in U.S. Pat. Nos. 3,472,931; 3,527,864; 3,896,238; 3,903,256; 3,952,099; 3,989,816; 4,046,886; 4,130,643; 4,130,667; 4,299,826; 4,335,115; 4,343,798; 4,379,454; 4,405,616; 4,746,515; 4,316,893; 4,405,616; 4,060,084; 4,379,454; 4,560,553; 4,863,952; 4,863,970; 4,879,275; 4,940,586; 4,960,771; 5,066,648; 5,164,406; 5,227,169; 5,229,130; 5,238,933; 5,308,625; 5,378,730; 5,420,106; 5,641,504; 5,716,638; 5,750,137; 5,785,991; 5,837,289; 5,834,468; 5,882,676; 5,912,009; 5,952,000; 6,004,578; and Idson, Percutaneous Absorption, J. Pharm. Sci. vol. 64, no. b6, June 1975, pp. 901-924, the disclosures of which are herein incorporated by reference.

The ranges of the components of the pharmaceutical composition may vary, but the active ingredients should be understood to add to 100 weight percent of the active pharmaceutical composition.

The present invention is also directed to methods of treating, preventing, or managing dermatological conditions comprising administering to a patient a therapeutically effective amount of a composition of the invention. Such methods are used for the prevention, treatment, or management of one or more dermatological conditions all while substantially avoiding irritation to the skin, scalp, hair, or nails. The term “therapeutically effective amount” means that amount of the pharmaceutical composition that provides a therapeutic benefit in the treatment, prevention, or management of one or more skin conditions. The compositions may be administered in high concentrations for administration as a cleanser to be removed shortly thereafter, as well as in lower concentrations that are safer for products that can remain in contact with the skin for longer times.

The magnitude of a prophylactic or therapeutic dose of the composition in the acute or chronic management of dermatological conditions will vary with the severity of the condition to be treated and the route of administration. The dose, and perhaps the dose frequency, will also vary according to the age, body weight, and response of the individual patient. In general, a preferred topical daily dose range, in single or divided doses, for the conditions described herein should be from about 1 mg to 20,000 mg, more preferably about 2,000 mg to 16,000 mg, and most preferably about 6,000 mg to 10,000 mg of the active components (i.e., excluding excipients and carriers).

Those of ordinary skill in the art will also understand that topical effectiveness of pharmaceuticals requires percutaneous absorption and bioavailability to the target site. Thus, the compositions and methods of the invention require penetration through the stratum corneum into the epidermal layers of the skin or scalp or penetration into the surface of the hair or nails, as well as sufficient distribution to the sites targeted for pharmacologic action.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

The term “unit dose” is meant to describe a single dose, although a unit dose may be divided, if desired. About 1 to 2 unit doses of the present invention are typically administered per day, preferably about 1 dose per day.

Desirably, each unit dose, e.g., gel, cream, or ointment, contains from about 1 mg to 2,000 mg of the active ingredient, preferably about 200 mg to 1,600 mg, and more preferably about 600 mg to 1,000 mg of the composition Topical administration is preferred for the compositions and methods of the invention. Suitable dosage forms include dispersions, suspensions, solutions, aerosols, sponges, cotton applicators, and the like, with topical dosage forms such as shampoos, creams, and gels being preferred.

The present invention may include components such as suspensions, solutions and elixirs. The pharmaceutical compositions used in the methods of the present invention include the active ingredients described above, and may also contain pharmaceutically acceptable carriers, excipients and the like, and optionally, other therapeutic ingredients.

Because of its ease of administration, a cream, lotion, or ointment represents the most advantageous topical dosage unit form, in which case liquid pharmaceutical carriers may be employed in the composition. These creams, lotions, or ointments, may be prepared as rinse-off or leave-on products, as well as two stage treatment products for use with other skin cleansing or managing compositions. In a preferred embodiment, the compositions are administered as a rinse-off product in a higher concentration form, such as a gel, and then a leave-on product in a lower concentration to avoid irritation of the skin, scalp, hair, or nails. Each of these forms is well understood by those of ordinary skill in the art, such that dosages may be easily prepared to incorporate the pharmaceutical composition of the invention.

Pharmaceutical compositions for use in the methods of the present invention suitable for topical administration may be presented as discrete units, each containing a predetermined amount of the active ingredient, including, but not limited to, aerosol sprays; powders; sticks; granules; creams (e.g., a conditioner); pastes; gels; lotions (e.g., a sunscreen); syrups; or ointments; which may be on sponges or cotton applicators or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the carrier(s) with the active ingredient, which constitutes one or more necessary ingredients. In general, the compositions are prepared by uniformly and intimately admixing the active ingredient with liquid carriers or finely divided solid carriers or both, and then, if necessary, shaping the product into the desired presentation.

The compositions of the invention may be administered in conjunction with one or more second dermatological compositions that may be administered by any suitable route of administration. Suitable routes of administration to provide the patient with an effective dosage of the one or more second dermatological compositions may be employed for of the other dermatological composition according to the methods of the present invention include, but are not limited to, oral, intraoral, rectal, parenteral, topical, epicutaneous, transdermal, subcutaneous, intramuscular, intranasal, sublingual, buccal, intradural, intraocular, intrarespiratory, or nasal inhalation and like forms of administration.

Suitable dosage forms for the one or more second dermatological compositions include, but are no limited to, tablets, troches, capsules, patches, gel caps, magmas, lozenges, plasters, discs, suppositories, nasal or oral sprays, and the like. When an oral dosage unit form is used, tablets, capsules, and gel caps are preferred, in which case solid pharmaceutical carriers may be employed. If desired, tablets may be coated by standard aqueous or nonaqueous techniques. For example, a tablet maybe prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

In addition to the common dosage forms set out above, the one or more second dermatological compositions for use in the methods of the present invention may also be administered by controlled release means and/or delivery devices such as those described in U.S. Pat. Nos. 3,845,770; 3,916,899; 3,536,809; 3,598,123; and 4,008,719, the disclosures of which are expressly incorporated herein by reference thereto.

### EXAMPLES

The invention is further defined by reference to the following examples describing in detail the preparation of the compound and the compositions used in the methods of the present invention, as well as their utility. The examples are representative, and they should not be construed to limit the scope of the invention.

### Example 1

### Skin Cleanser Formulation

A pharmaceutical composition according to the invention may be prepared for cleansing skin as set forth below:

| | Ingredient | Trade Name/Supplier | % by Weight |
|---|---|---|---|
| Part A | Deionized Water | N/A | 49.2 |
| | Trisodium Ethylene-Diamine- | HAMP-ENE Na₃T/Akzo Nobel | 0.2 |
| | Tetraacetic Acid (EDTA) | | |
| | Sodium Laureth-13 | SURFINE WLL/Finetex | 10 |
| | Carboxylate | | |
| | Disodium Laureth | MACKANATE EL/McIntyre | 17 |
| | Sulfosuccinate | Group | |
| | Disodium Cocoamphodiacetate | MONATERIC CDX-38/Mona | 11 |
| | PEG-150 Pentaerythrityl | CROTHIX/Croda | 1.5 |
| | Tetrastearate | | |
| | PEG-150 Distearate | KESSCO PEG 6000 DS/Stepan | .7 |
| | Methylparaben | N/A | 0.2 |
| Part B | Salicylic Acid | Salicylic Acid, powder, | 1.6 |
| | | USP/Spectrum | |
| | Citric Acid | N/A | 1.5 |
| | Triclosan | IRGASAN DP300/Ciba | 0.3 |
| Part C | PPG-26-Buteth-26, PEG-40 | SOLUBILISANT LR1/Les | 2 |
| | Hydrogenated Castor Oil | Colorant Wackherr SA | |
| | Fragrance (Parfum) | Fragrance - BELL #J7393/Bell | 0.3 |
| | | Flavors and Fragrances | |
| | Menthol | Menthol Crystals, USP | 0.1 |
| Part D | Butylene Glycol, Deionized | ACTIPHYTE OF BLACK | 0.1 |
| | water, Black Cohosh | SNAKEROOT BG50/Active | |
| | (Cimicifuga Racemosa) Extract | Organics | |
| | Butylene Glycol, Deionized | ACTIPHYTE OF JAPANESE | 0.1 |
| | water, Camellia Oleifera | GREEN TEA BG50/Active | |
| | Extract | Organics | |
| | Sodium Peroxylinecarbolic | AJIDEW-50/Ajinomoto | 0.2 |
| | Acid (PCA) | | |
| | Cocamidopropyl PG-Dimonium | PHOSPHOLIPID PTC/Mona | 1 |
| | Chloride Phosphate | | |
| Part E | Hydrogen Peroxide | Hydrogen Peroxide, 35% | 3 |
| | | solution, technical | |
| | | | 100% |
| HAMP-ENE Na₃T is commercially available from Akzo Nobel Inc. of Dobbs Ferry, NY; SURFINE WLL is commercially available from Finetex, Inc. of Elmwood Park, NJ; MACKANATE EL is commercially available from McIntyre Group of University Park, IL; MONATERIC CDX-38 and | | | |
| # PHOSPHOLIPID PTC are commercially available from Mona Industries Inc. of Patterson, NJ; CROTHIX is commercially available from Croda Inc. of Parsippany, NJ; KESSCO PEG 600 DS is commercially available from Stepan Co. of Northfield, IL; IRGASAN DP300 is commercially available | | | |
| # from Ciba Specialty Chemicals Corp. of Albemarle, NC; SOLUBILISANT LR1 is commercially available from Les Colorant Wackherr SA of St. Ouen L'Aumone, France; BELL #J7393 is commercially available from Bell Flavors and Fragrances of Northbrook, IL; ACTIPHYTE OF BLACK | | | |
| # SNAKEROOT BG50 and ACTIPHYTE OF JAPANESE GREEN TEA BG50 are commercially available from Active Organics of Dallas, TX; and AJIDEW-50 is commercially available from Ajinomoto USA Inc. of Teaneck, NJ. | | | |

Deionized water was metered into the processing tank and mixing subsequently begun. The water was heated to 75° C. and the remainder of Part A was added and mixed until uniform. The mixture was cooled to 60° C. and the Part B ingredients were added and mixed until uniform. The mixture was then cooled to 50° C. In a separate vessel, Part C was premixed until uniform and then added to the mixture of Parts A and B. Parts A, B, and C were mixed until uniform and cooled to 40° C. The Part D ingredients were added and mixed until uniform, then cooled to 30° C. Part E was added and mixed until uniform, resulting in a colorless, clear, slightly viscous fluid having a pH at 25° C. of between 4 to 4.5 and a viscosity between 3,000 to 4,000 cps (RVT: #4@10 rpm @25° C.).

### Example 2

### Advanced Acne Prone Skin Formulation

A pharmaceutical composition according to the invention may be prepared for treating skin prone to acne as set forth below:

| | Ingredient | Trade Name/Supplier | % by Weight |
|---|---|---|---|
| Part A | Deionized Water | N/A | 46.7 |
| | Hydroxyethylcellulose | CELLOSIZE | 1 |
| | | QP52,000H/Amerchol | |
| Part B | Tetrasodium Ethylene-Diamine- | HAMP-ENE 220/Akzo Nobel | 0.1 |
| | Tetraacetic Acid (EDTA) | | |
| | Butylene Glycol | 1,3-butylene glycol/Ashland | 5 |
| | Aloe Barbadensis Gel | Aloe Vera Freeze Dried | 0.1 |
| | | Powder 200:1/Aloe | |
| | Methyl Gluceth-10 | GLUCAM E-10/Amerchol | 3 |
| | Witch Hazel (Hamamelis | Witch Hazel Distillate, 14% | 3 |
| | Virginiana) Distillate | | |
| | Zinc Acetate | Zinc Acetate, crystals, | 0.5 |
| | | USP/FCC | |
| | Orange (Citrus Aurantium | NATURAL ORANGE | 0.3 |
| | Dulcis) ExtractMethylparaben | EXTRACT #71689/ | |
| | | Flavurence | |
| | Dipotassium Glycyrrhizate | N/A | 0.3 |
| | Lecithin, Tocopherol and | OXYSOMES/Barnett | 0.3 |
| | Magnesium Ascorbyl Phosphate | | |
| | Palmitoyl | GLYCOSPHERE PCO/Kobo | 0.2 |
| | Hydroxypropyltrimonium | | |
| | Amylopectin/Glycerin | | |
| | Crosspolymer, Lecithin, Grape | | |
| | (Vitis Vinifera) Seed Extract | | |
| | Palmitoyl | GLYCOSPHERE GT/Kobo | 0.5 |
| | Hydroxypropyltrimonium | | |
| | Amylopectin/Glycerin | | |
| | Crosspolymer, Lecithin, | | |
| | Camellia Sinensis Extract | | |
| | Epilobium Angustifolium | Canadian Willowherb Whole | 0.5 |
| | Extract | Extract (5% in | |
| | | water)/Fytokem | |
| | Butylene Glycol and Water and | ACTIPHYTE OF ARNICA | 0.5 |
| | Arnica Montana Extract | BG50/Active Organics | |
| Part C | Alcohol (denatured) | SD Alcohol 40-B, Anhydrous/ | 20 |
| | Salicylic Acid | Salicylic Acid, powder, | 1 |
| | | USP/FCC/Spectrum | |
| | Triclosan | IRGASAN DP300/Ciba | 0.4 |
| Part D | PPG-5-Ceteth-20 | PROCETYL AWS/Croda | 1 |
| | PEG-40 Hydrogenated Castor | CREMOPHOR RH-40/BASF | 0.6 |
| | Oil | | |
| | Retinol and Polysorbate 20 | RETINOL 50C/BASF | 0.1 |
| | Phytonadione | N/A | 0.1 |
| | Linoleic Acid | EMERSOL 315/Henkel | 0.3 |
| Part E | Glycolic Acid | GLYPURE = 70% Glycolic | 9 |
| | | Acid/DuPont | |
| Part F | Deionized water | N/A | 2 |
| | Sodium Hydroxide | Sodium Hydroxide, pellets, | 2 |
| | | USP/NF | |
| Part G | Hydrogen Peroxide | Hydrogen Peroxide, 35% | 1.5 |
| | | solution, technical | |
| | | | 100% |
| CELLOSIZE QP52,000H and GLUCAM E-10 are commercially available from Amerchol Corp. of Edison, NJ; HAMP-ENE 220 is commercially available from Akzo Nobel Inc. of Dobbs Ferry, NY; Aloe Vera Freeze Dried Powder 200:1 is commercially available from Aloe Corp. of TX; OXYSOMES | | | |
| # is commercially available from Barnet Products Corporation of Englewood Cliffs, NJ; Canadian Willowherb Whole Extract (5% in water) is commercially available from Fytokem, Inc. of Saskatoon, SK CANADA; GLYCOSPHERE PCO and GLYCOSPHERE GT are commercially available from Kobo | | | |
| # Products Inc. of South Plainfield, NJ; ACTIPHYTE OF ARNICA BG50 is commercially available from Active Organics of Dallas, TX; PROCETYL AWS is commercially available from Croda Inc. of Parsippany, NJ; CREMOPHOR RH-40 and RETINOL 50C are commercially available from BASF | | | |
| # Corporation of Budd Lake, NJ; GLYPURE = 70% Glycolic Acid is commercially available from DuPont of Wilmington, DE; EMERSOL 315 is commercially available from Henkel Corp. of Hoboken, NJ. | | | |

Deionized water was metered into the processing tank and mixing subsequently begun. CELLOSIZE QP52,000H was sprinkled in, heated to 70° C., and mixed until clear and uniform. The mixture was cooled to 40° C. Part B ingredients were added in the order above, with sufficient mixing after each ingredient was added. The mixture was cooled to 25° C. and premixed Part C ingredients were added and mixed until uniform. In a separate tank, Part D was heated to 40° C. until the solids were dissolved and then added to the batch of Parts A, B, and C. The mixture was mixed until uniform, then Part E was added and mixed until uniform. Premixed Part F was slowly added in increments as needed to obtain the desired pH of 3.3 to 3.8 at 25° C., then Part G was added and mixed until completely uniform. This resulted in a straw-colored, clear to slightly hazy, slightly viscous liquid having a pH (25° C. of 3.3 to 3.8 and a viscosity between 400 to 800 cps (RVT: #2@10 rpm@25° C.).

### Example 3

### Skin Perfecting Lotion

A pharmaceutical composition according to the invention may be prepared for treating skin as set forth below:

| | Ingredient | Trade Name/Supplier | % by weight |
|---|---|---|---|
| Part A | Water (Aqua) | Deionized water | 63.6 |
| | Carbomer | CARBOPOL ULTREZ 10/ | 0.3 |
| | | B. F. Goodrich | |
| | Sclerotium Gum | AMIGEL/Tri-K | 0.6 |
| | Glycerin | Glycerin 99.5%/Ashland | 6.0 |
| | Butylene Glycol | 1,3-butylene glycol/Ashland | 6.0 |
| | Allantoin | Allantoin/ISP | 0.6 |
| | Panthenol | DEXPANTHENOL/Roche | 0.6 |
| | Tetrasodium EDTA | HAMP-ENE 220/Akzo | 0.2 |
| | Methylparaben | Methylparaben/Ueno | 0.3 |
| | Sodium PCA | AJIDEW-50/Ajinomoto | 0.5 |
| Part B | Dicapryl Maleate | BERNEL ESTER DCM/Bernel | 6.0 |
| | Squalene | PHYTOLANE/Barnet | 0.8 |
| | Sorbitan Stearate | ARLACEL 60/ICI | 1.5 |
| | Stearic Acid | EMERSOL 132/Henkel | 1.3 |
| | Dimethicone | DOW CORNING 200, 350 | 0.8 |
| | | cs./Dow Corning | |
| | C12-C15 Alkyl Benzoate | FINSOLV TN/Finetex | 3.0 |
| | Cetearyl Alcohol and Ceteareth | HEXOTOL D/Heterene | 0.6 |
| | Propylparaben | Propylparaben/Ueno | 0.2 |
| Part C | Water (Aqua) | Deionized water | 0.3 |
| | Triethanolamine | Triethanolamine 99%/Ashland | 0.3 |
| Part D | Orange (Citrus Aurantium | NATURAL ORANGE | 0.3 |
| | Dulcis) Extract | EXTRACT #71689/Flavurence | |
| | Diazolidinyl Urea | GERMALL II/ISP | 0.3 |
| | Glycolipids and Hyaluronic Acid | PHYTO/CER HA/Tri-K | 0.3 |
| | Palmitoyl | GLYCOSPHERES PCO/Kobo | 0.3 |
| | Hydroxypropyltrimonium | | |
| | Amylopectin/Glycerin | | |
| | Crosspolymer and Lecithin and | | |
| | grape (Vitis Vinifera) Seed | | |
| | Extract | | |
| | Palmitoyl | GLYCOSPHERES GT/Kobo | 0.3 |
| | Hydroxypropyltrimonium | | |
| | Amylopectin/Glycerin | | |
| | Crosspolymer and Lecithin and | | |
| | Camellia Sinensis Extract | | |
| | Propylene Glycol | Propylene Glycol/Ashland | 0.6 |
| | Algae Extract | HAWAIIAN SEAPLANT | 0.2 |
| | | EXTRACT-J/Tri-K | |
| | Lecithin and Tocopherol and | OXYSOMES/Barnet | 0.6 |
| | Magnesium Ascorbyl Phosphate | | |
| | Butylene Glycol and Honey | ACTIPLEX 1072/Active | 1.1 |
| | Extract (Mel) and Meadowsweet | Organics | |
| | (Spiraea Ulmaria) Extract | | |
| | Talc and C9-C13 Fluoroalcohol | PF-5 TALC JA-46R/Kobo | 0.8 |
| | and Phosphoric Acid | | |
| | Hydrolyzed Soy Flour | RAFFERMINE/R.I.T.A. | 0.3 |
| | Oat (Avena Sativa) Protein | REDUCTINE/R.I.T.A. | 0.3 |
| | Phytonadione | Phytonadione/Roche | 0.01 |
| | Retinol and Polysorbate 20 | RETINOL 50C/BASF | 0.1 |
| | Epilobium Angustifolium Extract | Canadian Willowherb Whole | 0.5 |
| | | Extract (5% in water)/Fytokem | |
| | Arnica Montana Extract | ACTIPHYTE OF ARNICA | 0.5 |
| | | BG50/Active Organics | |
| | | | 100.0 |
| CARPOL ULTREZ 10 is commercially available from B. F. Goodrich Co. of Richfield, OH; AMIGEL, PHYTO/CER and HAWAIIAN SEA PLANT EXTRACT are available from Tri-K-Chemical of Fairview, MT; Allantoin and GERMALL II are available from ISP Chemicals Inc. of Calvert City, KY; | | | |
| # DEXPANTHENOL and Phytonadione are available from Roche Holdings, Inc. of Wilmington, DE; Methylparaben and Propylparaben are commercially available from Ueno Fine Chemicals Inc. of New York, NY AJIDEW N-50 is commercially available from Ajinomoto USA Inc. of Teaneck, NJ; | | | |
| # BERNEL ESTER is commercially available from Bernel Chemical Co. of Englewood, NJ; PHYTOLANE is commercially available from Barnet Products Corporation of Englewood Cliffs, NJ; ARLACEL 60 is commercially available from ICI Americas Inc. of Wilmington, DE; EMERSOL 132 is | | | |
| # commercially available from Henkel Corp. of Hoboken, NJ; DOW CORNING 200, 350 cs. is commercially available from Dow Corning Corp. of Auburn, MI; FINSOLV TN is commercially available from Finetex Inc. of Elmwood Park, NJ; HETOXOL D is commercially available from Heterene | | | |
| # Chemical Co. of Paterson, NJ; NATURAL ORANGE EXTRACT #71689 is commercially available from Flavurence Corp. of Annandale, NJ; ACTIPLEX 1072 is commercially available from Active Organics Inc. of Lewisville, TX; PF-5 TALC JA-46R is commercially available from Kobo | | | |
| # Products Inc. of South Plainfield, NJ; RAFFERMINE and REDUCTINE are commercially available from RITA Chemical Corp of East Northport, NY. | | | |

The Skin Perfecting Lotion was prepared by metering deionized water into a processing tank and mixing at high speed. CARBOPOL ULTREZ 10 was sprinkled in. When the CARBOPOL ULTREZ 10 was completely dispersed, AMIGEL was added and the mixture mixed until smooth and uniform. The mixture was heated to 80° C., the remaining Part A ingredients were added, and then mixed until uniform. In a separate tank, the Part B ingredients were combined and heated to 80° C. until all the solids were completely dissolved. Part B was added to Part A and the resulting batch was mixed until uniform. Premixed Part C was added and the batch mixed until homogeneous. The batch was cooled to 40° C. and the Part D ingredients were added and mixing continued until the temperature of the mixture was 35° C. The resulting Skin Perfecting Lotion was a light beige, opaque, viscous lotion having a pH at 25° C. of 6.2 to 7.2 and a viscosity of 14,000 to 24,000 cps. (RVT: #5@10 rpm@25° C.).

### Example 4

### Acne Management Formula

A pharmaceutical composition according to the invention may be prepared for managing acne as set forth below:

| | Ingredients | Trade Name/Supplier | % by weight |
|---|---|---|---|
| Part A | Water (Aqua) | Deionized Water | 56.8 |
| | Sclerotium Gum | AMIGEL/Alban Muller | 0.4 |
| | Disodium EDTA | HAM-ENE NA₂/Akzo | 0.3 |
| | Allantoin | Allantoin/ISP | 0.2 |
| | Methylparaben | Methylparaben/Ueno | 0.3 |
| | Zinc Oxide | 66 ZINC OXIDE | 0.3 |
| | | U.S.P./Whitaker, Clark & | |
| | | Daniels | |
| Part B | Water (Aqua) | Deionized Water | 10 |
| | Hydrolyzed Oat Flour and Oat | RITAVENA 5/R.I.T.A. | 2.8 |
| | Betaglucan | | |
| | Dicaprylyl maleate | BERNEL ESTER DCM/Bernel | 3 |
| | Glycerayl Stearate and PEG- | ARLACEL 165/ICI | 3 |
| | 100 Stearate | | |
| | Cetearyl Alcohol and | HEXOTOL D/Heterene | 3 |
| | Ceteareth-20 | | |
| | Propylparaben | Propylparaben/Ueno | 0.1 |
| Part D | Salicylic Acid | Salicylic Acid, powder, U.S.P.- | 1.3 |
| | | N.F./Spectrum | |
| | Sulfur | Sulfur, precipitated, U.S.P.- | 6.5 |
| | | N.F./Spectrum | |
| Part E | Water (Aqua) | Deionized Water | 3 |
| | Sodium Hydroxide | Sodium Hydroxide, pellets, | 0.1 |
| | | U.S.P.-N.F./Spectrum | |
| | Glycolic Acid | GLYPURE 70% GLYCOLIC | 6.5 |
| | | ACID/DuPont | |
| Part F | Orange (Citrus Aurantium | ORANGE EXTRACT | 1.1 |
| | Dulcis) Extract | PRODUCT #61522/Sunkist | |
| | Diazolidinyl Urea | GERMALL II/ISP | 0.4 |
| | Dipotassium Glycyrrhizate | Dipotassium | 0.3 |
| | | Glycyrrhizinate/Int'l Sourcing | |
| | Lecithin and Tocopherol and | OXYZOMES/Barnett | 0.3 |
| | Magnesium Ascorbyl | | |
| | Phosphate | | |
| | Palmitoyl | GLYCOSPHERES PCO/Kobo | 0.3 |
| | Hydroxypropyltrimonium | | |
| | Amylopectin/Glycerin | | |
| | Crosspolymer and Lecithin and | | |
| | Grape (Vitis Vinifera) Seed | | |
| | Extract | | |
| | | | 100.0 |
| AMIGEL is commercially available from Alban Muller International of Vincennes, France; HAM-ENE NA₂ is commercially available from Akzo Chemicals Inc. of Deer Park, TX; 66 ZINC OXIDE U.S.P. is commercially available from Whitaker, Clark & Daniels of South | | | |
| # Plainfield, NJ; Salicylic Acid, powder, U.S.P.-N.F., Sulfur, precipitated, U.S.P.-N.F. and Sodium Hydroxide, pellets, U.S.P.-N.F. are commercially available from Spectrum Mfg. Corp of New Brunswick, NJ; ORANGE EXTRACT PRODUCT #61522 is commercially available | | | |
| # from Sunkist Growers, Inc. of Van Nuys, CA; Dipotassium Glycyrrhizinate is commercially available from International Sourcing Inc. of Upper Saddle River, NJ. | | | |

The Acne Management Formula was prepared by metering deionized water into a processing tank and mixing at high speed. AMIGEL was sprinkled in. When the AMIGEL was completely dispersed, the mixture was heated to 85° C. and the remaining Part A ingredients were added and the mixture mixed well after each addition. In a separate tank, Part B was heated to 100° C., mixed until smooth, cooled to 80° C. and added to the batch. The resulting batch was mixed well. In another tank, the Part C ingredients were heated to 75° C. When all the solids dissolved, Part C was added to the batch, the batch was mixed until smooth and uniform, and the batch cooled to 50° C. Part D ingredients were added to the batch, the batch was homogenized for 5 to 10 minutes until the batch was smooth and uniform, and the batch was cooled to 40° C. The deionized water of part E was premixed with the sodium hydroxide pellets and the resulting solution was mixed well until all solids were dissolved. While mixing the solution, glycolic acid was slowly added in increments and the solution was mixed until homogeneous. The solution was added to the batch and the Part F ingredients were added to the batch. The batch was mixed and cooled to 35° C. The Acne Management Formula was a light yellow, opaque smooth lotion having a pH at 25° C. of 3.8 to 4.8 and a viscosity of 10,000 to 20,000 cps. (RVT: #5@10 rpm@25° C.).

### Example 5

### Clarifying Skin Cleanser

A pharmaceutical composition according to the invention may be prepared for managing acne as set forth below:

| | Ingredients | Trade Name/Supplier | % by weight |
|---|---|---|---|
| Part A | Water (Aqua) | Deionized Water | 48.5 |
| | Sodium Lauroyl Oat Amino | PROTEOL O.A.T./Seppic | 2 |
| | Acid | | |
| | Decyl Glucoside | ORAMIX NS-10/Seppic | 3 |
| | Cocamidopropyl Betaine | AMPHOSOL CA/Stephan | 12.5 |
| | Disodium Laureth | MACKANATE EL/McIntyre | 24 |
| | Sulfosuccinate | | |
| | PEG-120 Methyl Glucose | GLUCAMATE DOE- | 3.5 |
| | Dioleate | 120/Amerchol | |
| | Methylparaben | Methylparaben/Ueno | 0.2 |
| | PEG-150 Pentaerythrityl | CROTHIX/Croda | 0.25 |
| | Tetrastearate | | |
| Part B | Salicylic Acid | Salicylic Acid, powder, | 2 |
| | | USP/Spectrum | |
| | Tetrasodium EDTA | HAMP-ENE-100/Akzo | 0.3 |
| | Triclosan | IRGASAN D300/Ciba Specialty | 0.2 |
| | | Chemicals | |
| Part C | PPG-26-Buteth-26 and PEG | SOLUBILISANT LRI/ | 2 |
| | 40Hydrogenated castor Oil | Whittaker, Clark & Daniels | |
| | Fragrance | Fragrance-BELL #J7393/Bell | 0.3 |
| | Menthol | Menthol Crystals, USP/ | 0.1 |
| | | Spectrum | |
| Part D | Butylene Glycol and water | ACTIPHYTE OF BLACK | 0.2 |
| | (aqua) and Black Cohosh | SNAKEROOT BG50/Active | |
| | (Cimicifuga Racemosa) Extract | Organics | |
| | Butylene Glycol and water | ACTIPHYTE OF JAPANESE | 0.2 |
| | (aqua) and Camellia Oleifera | GREEN TEA BG50/Active | |
| | Extract | Organics | |
| | Sodium PCA | AJIDEW N-50/Ajinomoto | 0.4 |
| | Imidazolidinyl Urea | GERMALL 115/ISP | 035 |
| | | | 100.0 |
| PROTEAL O.A.T. is commercially available from Seppic Inc. of Fairfield, NJ; AMPHOSOL CA is commercially available from Stephan Co. Inc. of Fort Lauderdale, FL; GLUCAMATE DOE-120 is commercially available from Amerchol Corp. of Edison, NJ; HAMP-ENE-100 is | | | |
| # commercially available from Akzo Nobel Inc. of Dobbs Ferry, NY; SOLUBILISANT LRI is commercially available from Whitaker, Clark & Daniels of South Plainfield, NJ; GERMALL 115 is commercially available from ISP Chemicals Inc. of Calvert City, KY. | | | |

The Clarifying Skin Cleanser was prepared by metering deionized water into a processing tank, mixing, and heating to 75° C. The part A ingredients were added and mixed until all the solids dissolved. The resulting mixture was cooled to 60° C. In a separate vessel the Part B ingredients were combined. The Part B ingredients were then added to Part A and the resulting batch was mixed until uniform. The resulting mixture was cooled to 50° C. In a separate vessel the Part C ingredients were mixed until uniform. The part C ingredients were added to the batch and the resulting batch was mixed until uniform. The batch was cooled to 40° C. and the part D ingredients were added and mixing continued until uniform followed by cooling to 30° C. The Clarifying Skin Cleanser Formula was a pale yellow, slightly viscous liquid having a pH at 25° C. of 4.5 to 5.5 and a viscosity of 5,000 to 9,000 cps. (RVT: #@10 rpm@25° C.).

### Example 6

### Antimicrobial Effectiveness of the Invention—Advanced Acne Prone Skin Formulation

Culture Preparation

Escherichia coli (ATCC #8739), Staphylococcus pureus (ATCC #6533), Pseudomonas aeruginosa (ATCC #9027) were each propagated in Trypicase Soy Broth (TSB) at 35° C. for 24 hrs. Candida albicans (ATCC #10231), and Aspergillus niger (ATOC #16404) were propagated in Yeast and Mold Broth (YM) at 24° C. for 72 h. One loop of each bacteria culture was streaked onto Trypticase Soy Agar (TSA) and the yeast and mold onto Sabouraud Dextrose Agar (SDA). The bacterial and yeast cultures were incubated for 24 h at 35° C. and 48 h at 24° C., respectively. The mold culture was incubated for 5 days at 24° C. Following appropriate incubation, the surface growth of the organisms were washed with sterile Saline TS. Additional saline was added to reduce the microbial count. Each respective cell suspension was further diluted with sterile saline TS to an appropriate concentration.

Product Inoculation

Five 20-g portions of the Advanced Acne Prone Skin Formula of Example 2 was aseptically placed into sterile bottles. Each bottle was independently inoculated with 0.1 mL of the inoculum suspension.

Target Inoculation Concentration

A final concentration of 10⁵ and 10⁶ cfu/g of product was obtained. This spike suspension was assayed for each respective organism to determine the initial microbial load in the product. All enumeration analyses were performed by preparing serial 10-fold dilutions in Butterfield's Phosphate Buffered Diluent (BPBD), and then plated using the pour plate technique on respective media.

Test Intervals

An enumeration of the target organisms were performed on each inoculum. Immediately after inoculation (less than 1 minute), each product was assayed to determine the density of viable target organisms according to the pour plate technique. Each sample was tested 4 minutes. A 1-g portion was removed and mixed with 9.9 mL of BPBD. Serial dilutions were prepared as appropriate. Test samples containing bacterial cultures were plated with TSA and incubated for 48 h at 35° C. Samples containing yeast and mold were plated with SDA and incubated for 5 days at 24° C.

Results

The following results were obtained for each of the five organisms.

Test Organism: Candida albicans (ATOC # 10231)
Theoretical Inoculum Level: 400,000 cfu/g

| Testing | |
|---|---|
| Schedule | Recovery Levels (cfu/g) |
| (Time: minutes) | Advanced Acne Prone Skin Formula |
| 0 (less than 1) | <10 |
| 2 | <10 |
| 4 | <10 |

Test Organism: Aspergillus niger (ATCC # 16404)
Theoretical Inoculum Level: 160,000 cfu/g

| Testing | |
|---|---|
| Schedule | Recovery Levels (cfu/g) |
| (Time: minutes) | Advanced Acne Prone Skin Formula |
| 0 (less than 1) | <10 |
| 2 | <10 |
| 4 | <10 |

Test Organism: Escherichia coli (ATCC # 8739)
Theoretical Inoculum Level: 1,000,000 cfu/g

| Testing | |
|---|---|
| Schedule | Recovery Levels (cfu/g) |
| (Time: minutes) | Advanced Acne Prone Skin Formula |
| 0 (less than 1) | <10 |
| 2 | <10 |
| 4 | <10 |

Test Organism: Staphylococcus aureus (ATCC # 6538)
Theoretical Inoculum Level: 700,000

| Testing | |
|---|---|
| Schedule | Recovery Levels (cfu/g) |
| (Time: minutes) | Advanced Acne Prone Skin Formula |
| 0 (less than 1) | <10 |
| 2 | <10 |
| 4 | <10 |

Test Organism: Pseudomonas aeruginosa (ATCC # 9027)
Theoretical Inoculum Level: 260,000

| Testing | |
|---|---|
| Schedule | Recovery Levels (cfu/g) |
| (Time: minutes) | Advanced Acne Prone Skin Formula |
| 0 (less than 1) | <10 |
| 2 | <10 |
| 4 | <10 |

Discussion and Conclusion

The Advanced Acne Prone Skin Formulation prepared according to the present invention exhibited excellent antimicrobial properties. In less than one minute there was greater than a 99.99% reduction in levels of Candida albicans, Escherichia coli, Staphylococcus aureus, Pseudomonas aeruginosa, and Aspergillus niger.

### Example 7

### Antimicrobial Effectiveness of Another Formulation of the Invention—Clarifying Skin Cleanser

Culture Preparation

Escherichia coli (ATCC #8739), Staphylococcus pureus (ATCC #6533), and Pseudomonas aeruginosa (ATCC #9027) were propagated in Trypicase Soy Broth (TSB) at 35° C. for 24 h. Candida albicans (ATCC #10231) and Aspergillus niger (ATOC #16404) were propagated in Yeast and Mold Broth (YM) at 24° C. for 72 h. One loop of each bacteria culture was streaked onto Trypticase Soy Agar (TSA) and the yeast and mold onto Sabouraud Dextrose Agar (SDA). The bacterial and yeast cultures were incubated for 24 h at 35° C. and 48 h at 24° C., respectively. The mold culture was incubated for 5 days at 24° C. Following appropriate incubation, the surface growth of the organisms were washed with sterile Saline TS. Additional saline was added to reduce the microbial count. Each respective cell suspension was further diluted with sterile saline TS to an appropriate concentration.

Product Inoculation

Five 20-g portions of the Clarifying Skin Cleanser of Example 1 was aseptically placed into sterile bottles. Each bottle was independently inoculated with 0.1 mL of the inoculum suspension.

Target Inoculation Concentration

A final concentration of 10⁵ and 10⁶ cfu/g of product was obtained. This spike suspension was assayed for each respective organism to determine the initial microbial load in the product. All enumeration analyses were performed by preparing serial 10-fold dilution's in Butterfield's Phosphate Buffered Diluent (BPBD), and then plated using the pour plate technique on respective media.

Test Intervals

An enumeration of the target organisms were performed on each inoculum. Immediately after inoculation (less than 1 minute), each product was assayed to determine the density of viable target organisms according to the pour plate technique. Each sample was tested again after 2 and 4 minutes. A 1-g portion was removed and mixed with 9.9 mL of BPBD. Serial dilutions were prepared as appropriate. Test samples containing bacterial cultures were plated with TSA and incubated for 48 h at 35° C. Samples containing yeast and mold were plated with SDA and incubated for 5 days at 24° C.

Results

The following results were obtained for each of the five organisms.

Test Organism: Candida albicans (ATOC # 10231)
Theoretical Inoculum Level: 400,000 cfu/g

| Testing | |
|---|---|
| Schedule | Recovery Levels (cfu/g) |
| (Time: minutes) | Clarifying Skin Cleanser |
| 0 (less than 1) | 25,000 |
| 2 | 20,000 |
| 4 | 14,000 |

Test Organism: Aspergillus niger (ATCC # 16404)
Theoretical Inoculum Level: 160,000 cfu/g

| Testing | |
|---|---|
| Schedule | Recovery Levels (cfu/g) |
| (Time: minutes) | Clarifying Skin Cleanser |
| 0 (less than 1) | 1,400 |
| 2 | 1,200 |
| 4 | 1,000 |

Test Organism: Escherichia coli (ATCC # 8739)
Theoretical Inoculum Level: 1,000,000 cfu/g

| Testing | |
|---|---|
| Schedule | Recovery Levels (cfu/g) |
| (Time: minutes) | Clarifying Skin Cleanser |
| 0 (less than 1) | <10 |
| 2 | <10 |
| 4 | <10 |

Test Organism: Staphylococcus aureus (ATCC # 6538)
Theoretical Inoculum Level: 700,000

| Testing | |
|---|---|
| Schedule | Recovery Levels (cfu/g) |
| (Time: minutes) | Clarifying Skin Cleanser |
| 0 (less than 1) | <10 |
| 2 | <10 |
| 4 | <10 |

Test Organism: Pseudomonas aeruginosa (ATCC # 9027)
Theoretical Inoculum Level: 260,000

| Testing | |
|---|---|
| Schedule | Recovery Levels (cfu/g) |
| (Time: minutes) | Clarifying Skin Cleanser |
| 0 (less than 1) | <10 |
| 2 | <10 |
| 4 | <10 |

Discussion and Conclusion

The Clarifying Skin Cleanser exhibited excellent antimicrobial properties. In less than one minute there was a >99.99% reduction in levels of Escherichia coli, Staphylococcus aureus, and Pseudomonas aeruginosa. In less than one minute, levels of Aspergillus niger and Candida albicans were reduced by 99.1% and 94.0%, respectively.

### Example 8

### Irritation Test Using the Invention

Irritation potential following epidermal contact by compositions prepared according to the invention was examined. Fifty-three subjects ranging from 18 to 77 were evaluated. The patients were administered 0.2 mL, or an amount sufficient to cover the upper back between the scapulae, of a 10 percent dilution of the formulation used in Example 2. The administration occurred by applying the composition to a 1″×¾″ absorbent pad portion of an adhesive dressing, which was secured to the treatment site on each patient. The test material remained in contact for a total of 48 hours, and the test sites were evaluated at that time and at 72 hours (24 hours later) for changes using a 6-point scale ranging from no visible skin reaction up to severe erythema, possible edema, vesiculation, bullae and/or ulceration. One test subject did not complete the study. Observations indicated negative irritation throughout the test interval, i.e. no visible skin reaction on a single patient.

### Example 9

### Hydrogen Peroxide Stability Test

The formulations prepared according to Examples 1 of the invention having hydrogen peroxide, citric acid, salicylic acid, an antibacterial agent, and an amphoteric surfactant were heated to between 40° C. to 45° C. for three months in an oven test. The oxygen content of the formula which was assayed after the stability test, showed no more than 3 weight percent loss of the original hydrogen peroxide content. Such high stability provides an improved composition having a long shelf-life without substantial loss of efficacy.

### Examples 10-12

### Acne Treatment Regimen

An acne treatment regimen comprising Clarifying Cleanser, Advanced Acne Prone Skin Formula, Skin Perfecting Lotion and Acne Management Formula (Examples 1, 2, 3, and 4, respectively) was administered to 15 subjects. Subjects were evaluated after 2 weeks and 4 weeks use of the treatment regimen. Subjects were evaluated for total facial lesions, skin hydration and overall appearance of acne.

Testing of the Treatment Regimen

The acne treatment regimen comprising a ADVANCED ACNE PRONE SKIN FORMULA, SKIN PERFECTING LOTION, ACNE MANAGEMENT FORMULA, and CLARIFYING SKIN CLEANSER, prepared according to Examples 2,3,4, and 5, respectively, was administered to 15 subjects who exhibited a Grade 2-4 acne condition according to the grading scale provided below:

0: Facial skin need not be perfectly clear. A few scattered comedones or papules may be present, but these should be visible only on close examination.

2: About one fourth of facial area is involved, with small papules and large or small comedones. A few pustules or large prominent papules may be present.

4: About half of facial area is involved, with small papules and large or small comedones. A few pustules or large prominent papules are usually present. (If lesions are large, subject may have Grade 4 severity, although less than half of facial area is involved).

6: About three-fourths of facial area is involved, with papules and/or large open comedones. (Lesser facial area of involvement is permissible if inflammatory lesions are large) numerous pustules are usually present, some of which may be large.

8: Practically all of facial area is involved, with lesions. Large prominent pustules are usually visible. Lesions are usually highly inflammatory. Other types of acne (such as conglobata, including sinus and cystic types).

On the first day of the study all subjects were acclimated to ambient temperature and relative humidity for fifteen minutes. After the equilibration period, a trained technician examined each subject's face and recorded the number of inflammatory and non-inflammatory lesions in each of six sections of the face. The lesions of the six sections were totaled to obtain a global assessment score for each subject. Clinical photographs were taken in various poses for each subject and three Corneometer measurements were taken.

Subjects were provided with the treatment regimen and were given the following instructions for the treatment regimen:

CLARIFYING CLEANSER: Apply twice per day (once in the morning and once in the evening). Pour a small amount into hand or wash cloth. Apply to dampened face and neck. Massage gently into full lather. Rinse thoroughly with warm water and pat dry. Follow with ACNE PRONE SKIN FORMULA.

ACNE PRONE SKIN FORMULA: Apply after cleansing twice per daily (once in the morning and once in the evening). Apply a small amount to face and neck or areas affected with acne. Follow with SKIN PERFECTING LOTION.

SKIN PERFECTING LOTION: Use twice per day after cleansing and treating skin. Apply a small amount to face and neck.

ACNE MANAGEMENT FORMULA: Use twice a day after using CLARIFYING CLEANSER, ACNE PRONE SKIN FORMULA, and SKIN PERFECTING LOTION. Apply a small amount to affected area to spot treat.

Subjects were required to maintain a daily diary indicating date, time of use and comments. Subjects were permitted to use their customary-make-up products during the study. However, subjects were instructed not to introduce any new cosmetic or facial treatment products during the study. Following the two week test material use period subjects were evaluated for an interim count of total facial lesions, Corneometer readings and clinical photographs. After four weeks of test material use subjects returned with their diaries for a final lesion count, Corneometer readings and clinical photographs. Standard paired t-tests were used to determine statistically significant differences between baseline and two (2) and four (4) week total facial lesion counts and Corneometer readings. Statistical significance exists for all p-values less than or equal to 0.05 at the 95% confidence level. Improvement scores for the appearance of acne in clinical photographs were analyzed using Z-tests.

A total of fourteen subjects finished the study. One subject was disqualified immediately for lack of compliance with the Inclusion Criteria of the protocol. A review of the daily diaries indicated that four (4) subjects reported redness, burning, stinging and/or “irritation” during the study period. One (1) of the subjects reported the onset of redness and burning on day five (5) of the study immediately after product application and lasting for fifteen (15) to twenty (20) minutes. The subject was instructed to discontinue test material use on day ten (10) of the study. On day fourteen (14) the subject was examined by a doctor and no evidence of skin irritation was observed. The subject was instructed to begin use of the treatment material at this time. The subject reported no evidence of irritation until day twenty four (24) of the study and completed study participation. No evidence of irritation was observed at the final visit. The subjects reaction was diagnosed as dermatitis. The remaining subjects reported symptoms following one (1) to two (2) uses of the test material and completed study participation without further complaints.

### Example 10

### Total Lesion Count Following Treatment Regimen

The acne present on the skin of each subject was evaluated by visual examination using the grading scale described herein. The number of lesions on the face were counted at each visit. The number of open and closed comedones, as well as papules and pustules, were recorded. A global assessment score, the total of all lesions, was recorded for each visit. Reductions in the global assessment score are indicative of a reduced incidence and/or severity of acne lesions. The data for total lesion count is provided below.

Total Lesion Count

| | Baseline | 2 Weeks | 4 Weeks |
|---|---|---|---|
| Mean | 44.4 | 33.4 | 27.6 |

| | | |
|---|---|---|
| Mean Percent Difference from Baseline | −26% | −40% |
| σ | 30% | 22% |

The regimen showed a statistically significant decrease of twenty-six percent (26%) in the number of lesions observed after using the treatment regimen for two (2) weeks and a statistically significant decrease of forty (40%) after using the treatment regimen for four (4) weeks compared to baseline (p=0.02 and p=1.07 E-05, respectively).

### Example 11

### Photographic Evaluation Following Treatment Regimen

Photographs of subjects were taken at designated visits using the Canfield Clinical System of imaging equipment. This particular system permits comparison of photographs to be made with the confidence that the only factors which may have changed are those resulting from treatment. This is achieved by precisely and reproducibly positioning the head of the subject and carefully controlling the lighting, film type and processing. Photographs were visually assessed and evaluated by a trained technician before and after use of the test material. The following scoring scale was used for visual assessment of the skin:

1=no improvement

2=slight improvement

3=mild improvement

4=moderate improvement

5=extreme improvement

Improvement scores for the appearance of acne in clinical photographs were analyzed using Z-tests. For the two (2) and four (4) week scores, the number of subjects exhibiting improvements scoring a two (2), three (3), four (4) or five (5) was compared to the number of subjects exhibiting no improvement, scored as a one (1). The improvement assessment of the overall appearance of acne, rated from clinical photographs, is provided below.

Photographic Evaluation

| | Score: | 1 | 2 | 3 | 4 | 5 |
|---|---|---|---|---|---|---|
| Week 2 | Number of Subjects | 5 | 5 | 2 | 2 | 0 |
| | Assigned each Score | | | | | |
| | Percentage | 35.7% | 64.3% | | | |
| | Z-Score | −1.12 | | | | |
| Week 4 | Number of Subjects | 4 | 4 | 5 | 1 | 0 |
| | Assigned each Score | | | | | |

| | | |
|---|---|---|
| Percentage | 28.6% | 71.4% |

| | |
|---|---|
| Z-Score | −1.77 |

The number of subjects exhibiting improvement from baseline in the overall appearance of acne at two (2) weeks was greater than subjects with no improvement. The Z-score obtained at two (2) weeks corresponds to improved skin appearance having a statistical significance at a 74% confidence level. The four (4) week photograph the number of subjects exhibiting improvement from baseline in the overall appearance of acne was greater than subjects with no improvement. The Z-score obtained at four (4) weeks corresponds to improved skin appearance having statistical significance at a 92% confidence level.

### Example 12

### Moisturization via Corneometer Following Treatment Regimen

Changes in skin hydration were measured with a CORNEOMETER which is a commercially available instrument (CM-820, Courage and Khazaka Germany) designed to measure changes in the capacitance of the skin resulting from small changes in the degree of hydration. The CORNEOMETER expresses the capacitance of the skin in arbitrary unit of skin hydration (H). The instrument is capable of measuring the moisture of the stratum corneum to a depth of 0.1 mm and is used to measure the effects of cosmetic preparations on the moisture content of the skin. Tests using the CORNEOMETER were conducted by taking 3 measurements, one at the right and left cheek and one at the center of the skin, for each subject.

The three measurements were then averaged for each subject. The data for skin hydration (H) is provided below.

Skin Hydration (H)

| | Baseline | 2 Weeks | 4 Weeks |
|---|---|---|---|
| Mean | 70.8 | 51.6 | 49.5 |

| | | |
|---|---|---|
| Mean Percent Difference from Baseline | −26% | −29% |
| σ | 14% | 12% |

The regimen showed a statistically significant decrease in Skin Hydration, H, of twenty-six percent (26%) after using the treatment regimen for two (2) weeks and a statistically significant decrease of twenty-nine (29%) after using the treatment regimen four (4) weeks compared to baseline (p=2.27 E-05 and p=5.38 E-06, respectively). A loss in skin hydration is typically observed following treatment with anti-acne products.

### Example 13

### Skin Cleanser of Invention with Antifungal and Antibacterial Agents

A pharmaceutical composition according to the invention may be prepared for cleansing skin as set forth below:

| | Ingredient | Trade Name/Supplier | % by Weight |
|---|---|---|---|
| Part A | Deionized Water | N/A | 50 |
| | Trisodium Ethylene-Diamine- | HAMP-ENE Na₃T/Akzo Nobel | 0.2 |
| | Tetraacetic Acid (EDTA) | | |
| | Sodium Laureth-13 | SURFINE WLL/Finetex | 10 |
| | Carboxylate | | |
| | Disodium Laureth | MACKANATE EL/McIntyre | 17 |
| | Sulfosuccinate | Group | |
| | Disodium Cocoamphodiacetate | MONATERIC CDX-38/Mona | 11 |
| | PEG-150 Pentaerythrityl | CROTHIX/Croda | 1.5 |
| | Tetrastearate | | |
| | PEG-150 Distearate | KESSCO PEG 6000 DS/Stepan | 0.7 |
| | Methylparaben | N/A | 0.2 |
| Part B | Clotrimazole | N/A | 0.8 |
| | Citric Acid | N/A | 1.5 |
| | Triclosan | IRGASAN DP300/Ciba | 0.3 |
| Part C | PPG-26-Buteth-26, PEG-40 | SOLUBILISANT LR1/Les | 2 |
| | Hydrogenated Castor Oil | Colorant Wackherr SA | |
| | Fragrance (Parfum) | Fragrance - BELL #J7393/Bell | 0.3 |
| | | Flavors and Fragrances | |
| | Menthol | Menthol Crystals, USP | 0.1 |
| Part D | Butylene Glycol, Deionized | ACTIPHYTE OF BLACK | 0.1 |
| | water, Black Cohosh | SNAKEROOT BG50/Active | |
| | (Cimicifuga Racemosa) Extract | Organics | |
| | Butylene Glycol, Deionized | ACTIPHYTE OF JAPANESE | 0.1 |
| | water, Camellia Oleifera | GREEN TEA BG50/Active | |
| | Extract | Organics | |
| | Sodium Peroxylinecarbolic | AJIDEW-50/Ajinomoto | 0.2 |
| | Acid (PCA) | | |
| | Cocamidopropyl PG-Dimonium | PHOSPHOLIPID PTC/Mona | 1 |
| | Chloride Phosphate | | |
| Part E | Hydrogen Peroxide | Hydrogen Peroxide, 35% | 3 |
| | | solution, technical | |
| | | | 100% |

Deionized water was metered into the processing tank and mixing subsequently begun. The water was heated to 75° C. and the remainder of Part A was added and mixed until uniform. The mixture was cooled to 60° C. and the Part B ingredients were added and mixed until uniform. The mixture was then cooled to 50° C. In a separate vessel, Part C was premixed until uniform and then added to the mixture of Parts A and B. Parts A, B, and C were mixed until uniform and cooled to 40° C. The Part D ingredients were added and mixed until uniform, then cooled to 30° C. Part E was added and mixed until uniform, resulting in a colorless, clear, slightly viscous fluid having a pH at 25° C. of between 4 to 6 and a viscosity between 3,000 to 4,000 cps (RVT: #4 (10 rpm@25° C.).

### Example 14

### Skin Cleanser of Invention with Antifungal and Antibacterial Agents

A pharmaceutical composition according to the invention maybe prepared for cleansing skin as set forth below:

| | Ingredient | Trade Name/Supplier | % by Weight |
|---|---|---|---|
| Part A | Deionized Water | N/A | 50 |
| | Trisodium Ethylene-Diamine- | HAMP-ENE Na₃T/Akzo Nobel | 0.2 |
| | Tetraacetic Acid (EDTA) | | |
| | Sodium Laureth-13 | SURFINE WLL/Finetex | 10 |
| | Carboxylate | | |
| | Disodium Laureth | MACKANATE EL/McIntyre | 17 |
| | Sulfosuccinate | Group | |
| | Disodium Cocoamphodiacetate | MONATERIC CDX-38/Mona | 11 |
| | PEG-150 Pentaerythrityl | CROTHIX/Croda | 1.5 |
| | Tetrastearate | | |
| | PEG-150 Distearate | KESSCO PEG 6000 DS/Stepan | .7 |
| | Methylparaben | N/A | 0.2 |
| Part B | Ciclopirox Olamine | N/A | 0.8 |
| | Citric Acid | N/A | 1.5 |
| | Triclosan | IRGASAN DP300/Ciba | 0.3 |
| Part C | PPG-26-Buteth-26, PEG-40 | SOLUBILISANT LR1/Les | 2 |
| | Hydrogenated Castor Oil | Colorant Wackherr SA | |
| | Fragrance (Parfum) | Fragrance-BELL #J7393/Bell | 0.3 |
| | | Flavors and Fragrances | |
| | Menthol | Menthol Crystals, USP | 0.1 |
| Part D | Butylene Glycol, Deionized | ACTIPHYTE OF BLACK | 0.1 |
| | water, Black Cohosh | SNAKEROOT BG50/Active | |
| | (Cimicifuga Racemosa) Extract | Organics | |
| | Butylene Glycol, Deionized | ACTIPHYTE OF JAPANESE | 0.1 |
| | water, Camellia Oleifera | GREEN TEA BG50/Active | |
| | Extract | Organics | |
| | Sodium Peroxylinecarbolic | AJIDEW-50/Ajinomoto | 0.2 |
| | Acid (PCA) | | |
| | Cocamidopropyl PG-Dimonium | PHOSPHOLIPID PTC/Mona | 1 |
| | Chloride Phosphate | | |
| Part E | Hydrogen Peroxide | Hydrogen Peroxide, 35% | 3 |
| | | solution, technical | |
| | | | 100% |

Deionized water was metered into the processing tank and mixing subsequently begun. The water was heated to 75° C. and the remainder of Part A was added and mixed until uniform. The mixture was cooled to 60° C. and the Part B ingredients were added and mixed until uniform. The mixture was then cooled to 50° C. In a separate vessel, Part C was premixed until uniform and then added to the mixture of Parts A and B. Parts A, B, and C were mixed until uniform and cooled to 40° C. The Part D ingredients were added and mixed until uniform, then cooled to 30° C. Part E was added and mixed until uniform, resulting in a colorless, clear, slightly viscous fluid having a pH at 25° C. of between 4 to 6 and a viscosity between 3,000 to 4,000 cps (RVT: #4 (10 rpm@25° C.).

Various modifications of the invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

What is claimed is:

1. A pharmaceutical composition for treating, preventing, or managing a dermatological condition selected from the group consisting of a scalp condition, a hair condition, and a nail condition comprising:
hydrogen peroxide in an amount sufficient to cleanse a dermatological surface without substantial irritation thereof;
a moisturizing agent in an amount sufficient to facilitate hydration of the dermatological surface or prevent moisture loss from the dermatological surface; and
one or more dermatological agents selected from an antimicrobial agent in an amount sufficient to inhibit microorganisms on the scalp, hair, or nails; an anti-inflammatory agent in an amount sufficient to reduce inflamation of the scalp, hair, or nails; or a combination thereof.

2. The pharmaceutical composition of claim 1, wherein the dermatological condition is a scalp condition.

3. The pharmaceutical composition of claim 1, wherein the dermatological condition is a hair condition.

4. The pharmaceutical composition of claim 1, wherein the dermatological condition is a nail condition.

5. The pharmaceutical composition of claim 1, wherein the moisturizing agent is a hydrophobic agent, hydrophilic agent, or a combination thereof.

6. The pharmaceutical composition of claim 1, wherein the antimicrobial agent is selected from an antibacterial, antifungal, antiviral, an anthelmintic, or combinations thereof.

7. Thepharmaceutical composition of claim 6, wherein the antimicrobial is a single broad spectrum antimicrobial agent selected from echinacea, golden seal, benzalkonium chloride, benzethonium chloride, iodine, grape seed extract, pomegranate extract, green tea extract, polyphenols, and combinations thereof.

8. The pharmaceutical composition of claim 6, wherein the antibacterial is selected from triclosan, neomycin, polymyxin, bacitracin, clindamycin, benzoyl peroxide, a tetracycline, a sulfa drug, a penicillin, a quinolone, a cephalosporin, and combinations thereof.

9. The pharmaceutical composition of claim 6, wherein the antiviral is selected from acyclovir, tamivir, penciclovir, and combinations thereof.

10. The pharmaceutical composition of claim 6, wherein the antifungal is selected from farnesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and combinations thereof.

11. The pharmaceutical composition of claim 6, wherein the anthelmintic is metronidazole.

12. The pharmaceutical composition of claim 1, wherein the anti-inflammatory is a non-steroidal agent, a steroidal agent, or a combination thereof.

13. The pharmaceutical composition of claim 12, wherein the steroidal agent is selected from hydrocortisone, fluocinolone acetonide, halcinonide, halobetasol propionate, clobetasol propionate, betamethasone dipropionate, betamethasone valerate, triamcinolone acetonide, and combinations thereof.

14. The pharmaceutical composition of claim 12, wherein the non-steroidal agent is selected from aspirin, ibuprofen, ketoprofen, naproxen, aloe vera gel, aloe vera, licorice extract, pilewort, Canadian willow root, zinc, allantoin, and combinations thereof.

15. The pharmaceutical composition of claim 1, further comprising a pharmaceutically acceptable carrier or excipient.

16. The pharmaceutical composition of claim 1, further comprising an exfoliant.

17. The pharmaceutical composition of claim 16, wherein the exfoliant is an enzymatic exfoliant.

18. The pharmaceutical composition of claim 16, wherein the exfoliant is an acidic exfoliant.

19. The pharmaceutical composition of claim 1, further comprising an amount of amphoteric surfactant and an amount of citric acid sufficient to inhibit hydrogen peroxide decomposition for at least three months.

20. The pharmaceutical composition of claim 19, wherein the amount of amphoteric surfactant and citric acid is sufficient to inhibit hydrogen peroxide decomposition at 40° C. for at least three months.

21. The pharmaceutical composition of claim 1, wherein the hydrogen peroxide is present in an amount from about 0.01 to 6 weight percent of the composition and the moisturizing agent is present in an amount from about 0.01 to 20 weight percent of the composition.

22. The pharmaceutical composition of claim 1, further comprising an amount of Retinol, Vitamin K, Arnica Montana, an immuno-enhancer, anti-oxidant, an extract of pomegranate, an extract of Morinda citrifolia, and combinations thereof.

23. A method of treating, preventing, or managing a dermatological condition selected from the group consisting of a scalp condition, a hair condition, and a nail condition in a patient which comprises administering to the patient a therapeutically effective amount of a composition comprising:
hydrogen peroxide in an amount sufficient to cleanse the surface of the scalp, hair, or nails without substantial irritation thereof; a moisturizing agent in an amount sufficient to facilitate hydration of the dermatological surface or prevent moisture loss from the dermatological surface; and
one or more dermatological agents selected from an antimicrobial agent in an amount sufficient to at least inhibit microorganisms on the scalp, hair, or nails; an anti-inflammatory agent in an amount sufficient to reduce inflamation of the scalp, hair, or nails; or a combination thereof for treating, preventing, or managing the dermatological condition.

24. The method of claim 23, wherein the antimicrobial agent is selected from an antibacterial, antifungal, antiviral, an anthelmintic, and combinations thereof.

25. The method of claim 23, wherein the hydrogen peroxide, the moisturizing agent, and the one or more dermatological agents are administered topically.

26. The method of claim 25, wherein the topical administration is by a gel, paste, cream, lotion, emulsion, shampoo, or ointment.

27. The method of claim 23, wherein the hydrogen peroxide, the moisturizing agent, and the one or more dermatological agents are administered concurrently.

28. The method of claim 23, wherein the hydrogen peroxide, the moisturizing agent, and the one or more dermatological agents are administered concurrently with at least one additional pharmaceutical composition for the prevention, treatment, or management of a dermatological condition.

29. The method of claim 28, wherein the at least one additional pharmaceutical composition is selected from yellow dock, bupleurum, poria cocos, gentian root, myrr gum, hawthorn berry extract, rosemary extract, wild yam root, wild yam extract, marshmallow root, black cohosh, soy, ginger, an anti-oxidant, an immuno-enhancer, and combinations thereof.

30. The method of claim 23, wherein the dermatological condition is at least one of psoriasis, folliculitis, rosacea, nail fungus, perioral dermatitis, seborrheic dermatitis, dandruff, or impetigo.

31. The method of claim 23, wherein about 1 mg to 20,000 mg of the composition is administered.
